(19) United States
(12) Patent Application Publication
(10) Pub. No.: US 2011/0046006 A1
(43) Pub. Date: Feb. 24, 2011

(54) MEANS AND METHODS FOR TYPING A CELL ISOLATE OF AN INDIVIDUAL SUFFERING FROM A PSYCHIATRIC DISORDER OR AT RISK OF SUFFERING THERE FROM

(76) Inventors: Sabine Spijker, Almere (NL); August Benjamin Smit, Amsterdam (NL); Witte Jonathan Gustav Hoogendijk, Amsterdam (NL)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/808,642

(22) PCT Filed: Dec. 19, 2008
(86) PCT No.: PCT/NL08/50837

§ 371 Date: Nov. 15, 2010

(30) Foreign Application Priority Data
Dec. 19, 2007 (EP) 07150177.9

(51) Int. Cl. C40B 30/04; C12Q 1/68; C07H 21/04; C40B 40/06

(52) U.S. Cl. 506/9; 435/6; 536/24.33; 506/16

(57) ABSTRACT

The present invention relates to a method for typing a cell isolate of an individual suffering from a pychiatric disorder, or at risk for suffering there from, the method comprising providing an RNA sample from a cell isolate from said individual; determining RNA levels for a set of genes in said RNA sample, wherein said set of genes comprises at least two of the genes listed in Table 9; and typing said isolate on the basis of the levels of RNA determined for said set of genes.

The invention relates to the field of medicine and to the field of diagnostics. The invention in particular relates to means and methods for typing cells and cell isolates from individuals suffering from, or at risk of suffering from, a psychiatric disorder, particularly a depressive disorder.

The invention is exemplified herein below predominantly for depressive disorders, however, this does not mean that the invention is limited thereto. The means and methods of the invention are suited for all psychiatric disorders, depressive disorders are nevertheless preferred. Depressive and anxiety disorders, have a high lifetime prevalence (16% and 10%, respectively) and frequently run a chronic course. There is considerable co-morbidity between depressive and anxiety disorders. They have many symptoms in common, similar pathogenic mechanisms are proposed and both disorders can be treated successfully with similar antidepressants. The pathogenesis of depressive and anxiety disorders is largely unknown but it is clear that genetic vulnerability and environmental risk factors are both important. At least two meta-analyses (Sullivan et al., 2000; Hettema et al., 2001) suggest that familial aggregation for depressive and anxiety disorders are due to genetic effects (heritability: depression 37%, panic disorder 43%, generalized anxiety disorder 32%), with a minimal contribution of environmental effects shared by family members, but substantial individual-specific environmental effects.

Depression has a large impact on human health (morbidity and mortality) and society in general and will be the second most important medical disorder by the year 2020, according to the World Health Organization. The three brain systems that are most strongly implicated in the pathogenesis of depression are: (i) the midbrain serotonin (5-HT) system; (ii) the medial prefrontal cortex (mPFC); and (iii) the hypothalamopituitary-adrenal (HPA) axis. Midbrain serotonergic neurons in the raphe nuclei participate in many physiological functions and are considered to be the cellular target of serotonin-specific reuptake inhibitors (SSRIs), the main class of drugs used to treat psychiatric illnesses. Their activity is controlled by 5-HT1A autoreceptors and by input from many brain areas, including the mPFC (see Hajós et al., 1998; Peyron et al., 1995). A disturbed 5-HT transmission of the raphe nuclei to their targets forms a plausible explanation for depression since SSRI's successfully normalise mood in many patients, whereas depletion of serotonin precursor tryptophan results in acute recurrence of depression (Artigas et al., 1996; Stahl, 2000; Blier and Bergeron, 1998; Haddjeri et al., 1998). In animal models, the 5-HT1A receptor was shown to act during development to establish normal anxiety-like behaviour in the adult (Gross et al., 2002). Relevance to humans is demonstrated by decreased binding of 5-HT1A tracers (Drevets et al., 2000; Sargent et al., 2000) and increased 5-HT1A autoreceptor density in the RN of depressed patients (Stockmeier et al., 1998). Polymorphisms of the serotonin transporter gene have also been associated with depression symptoms (Mann et al., 2000) and the occurrence of depression after stressful life-events (Caspi et al., 2003) or tryptophane depletion (Neumeister et al., 2002).

The prefrontal cortex regulates cognitive and associative functions and is involved in planning and execution of complex tasks (see Fuster, 1997). Depressed patients show hypoperfusion in mood related areas of the PFC (Drevets et al., 1997), which correlates with depression severity and normalises during treatment with SSRI (Soares and Mann, 1997). The mPFC is one of the few forebrain areas that projects densely to the serotonergic RN (Hajós et al., 1998; Peyron et al., 1995) and electrical stimulation of the mPFC indeed modulates the activity of 5-HT neurons (Hajós et al., 1998; Celada et al., 2001). In turn, the mPFC is densely innervated by serotonergic RN afferents (Azmitia and Segal, 1978). Especially layer V pyramidal neurons express 5-HT1A and 5-HT2A receptors (Pompeiano et al., 1994; Kia et al., 1996) and GABAergic interneurons express 5-HT2A and 5-HT3 receptors (see Jakab and Goldman-Rakic, 2000). The precise impact of 5-HT receptor activation in the mPFC microcircuits is currently unknown and may hold important clues to the understanding of depression. Hyperactivity of the HPA-axis is found in about 50% of depression patients. Successful antidepressive treatment often normalizes the HPA-axis (Inder et al., 2001) and corticotrophin releasing hormone (CRH)1-antagonists have antidepressant effects (Kunzel et al., 2003). Disturbed 5-HT transmission influences hypothalamic CRH producing neurons and the ensuing ACTH release from the pituitary and cortisol from the adrenal in animals and humans (Jorgensen et al., 2003). Furthermore, increased CRH gene expression in these hypothalamic targets of the serotonin system was observed in post mortem material of depressed patients (Raadsheer et al., 1995). Whether disturbed HPA axis activity is secondary to a disturbed 5-HT system or constitutes its own source of pathology remains an open question (see Kagamiishi et al., 2003; Montgomery et al., 2001; Oshima et al., 2003; Summers et al., 2003). In either case, the HPA axis provides a non-invasive read-out parameter, cortisol, which can be assessed in animals and in large scale human studies, by means of provocation tests (e.g. dexamethasone suppression test) and non-invasive salivary sampling (Kirschbaum and Hellhammer, 1989). Taken together, the evidence outlined above clearly identifies the raphe serotonin system, the mPFC and the HPA axis as central systems in the pathogenesis of the depression spectrum and emphasizes the evident interplay between the three systems.

Although much progress has been made in the field of depression, there is still much to be learned. Depression and anxiety disorders frequently run a chronic course, with a number of negative health care consequences, including increased medical consumption, disability, somatic morbidity and mortality. Given an extremely variable natural history, the most viable route for prevention appears to be to design ways to detect those at risk for an unfavourable prognosis in an early stage, tailoring interventions to the projected prognosis. A sobering finding, common to most psychiatric disorders, is that, until now, not one single determinant explains more than 15% of the aetiology of either disorder (McGuire and Troisi, 1998). Factors that have been identified are mostly very common, implying that they do not carry very high relative or absolute risks for incidence. Moreover, many risk factors (such as multiple loss) are not open to intervention. This severely limits the scope for primary and secondary prevention. Major depression is by far the most widely studied condition, concerning determinants of prognosis (for review, see Spijker et al., 2002). The predictive power of clinical factors, such as co-morbidity or duration and severity of earlier episodes, although limited, is hopeful, as these factors can be assessed in routine clinical work. However, these factors do not explain the wide variation in the prognosis. It is highly likely that underlying (molecular) biological factors determine both the clinical features of index episodes and (in interaction with environmental factors) their subsequent course. Moreover, individuals with similar depression-like symptoms can have very different clinical outcomes later in life. In fact many individuals suffer from depression-like symptoms at some point in their life but these are temporary and full recovery is possible without reverting to medication treatment. It is, at present, difficult to distinguish between groups that will recover without medication treatment and groups that do not recover without medication. This makes it difficult to determine the best treatment plan for individuals that present themselves with depression-like symptoms. Moreover, after a first depressive episode, about 50% of the patients will experience a recurrence of depression within a year. Therefore, physicians advise the patient according to protocol, to stay on antidepressant medication for six month after remission. However, most patients do not comply to this advice because it is difficult to take medication at a time there is no burden of disease, while adverse effects may be present. Furthermore, with our present knowledge we can not predict whether a particular patient belongs to the 50% in whom depression would indeed reoccur, when of medication. Finally, at present physicians can not predict which type of antidepressant (e.g. noradrenergic or serotonergic) will be effective for a particular patient, and have minimal adverse effects. The present invention relates to means and methods for typing and predicting: 1) which individuals with depression-like symptoms will become depressed when of medication; 2) which individuals with a depressive disorder will suffer from reoccurrence after premature termination of prophylactic antidepressant treatment; and 3) which patients benefit from what type of antidepressants, in terms of optimal effect and minimal adverse effect.

In particular the invention provides a method for typing a cell isolate of an individual suffering from a psychiatric disorder, or at risk of suffering there from, the method comprising providing an RNA sample from a cell isolate from said individual, determining RNA levels for a set of genes in said RNA sample, wherein said set of genes comprises at least two of the genes listed in Table 9, preferably at least two of the genes having the gene number 1-106 listed in Table 9 (first column), more preferably at least two of the genes having the gene number 1-142 listed in Table 9 (first column), and typing said isolate on the basis of the levels of RNA determined for said set of genes.

Part of the biological changes described above is probably caused by genetic polymorphisms (Flint at al., 1995; Holsboer et al., 1995). It is generally expected that in the following years a large number of polymorphisms will be discovered in candidate genes that code for proteins, which are already known to be involved in the pathogenesis of a psychiatric disease such as, for example, a depression (Merikangas, 2002). Twin studies have shown that depression is at least partly genetically determined, whereby genetic polymorphisms underlie biological factors which, in interaction with environmental factors, determine the course of a psychiatric disease such as a depressive disorder. A method according to the invention allows typing of a cell isolate from an individual based on RNA levels of a set of genes in said cell isolate. Without being bound by theory it is believed that at least some of the differences in gene expression detected using a method of the invention are due to differentially expressed polymorphic genes. These genes are typically associated with their most proximal gene product (i.e. gene-expression- and protein profiles). Polymorphic differences underlying a psychiatric disease such as a depressive disorder are at least to some extent reflected in differences in gene expression patterns present in a cell isolate. Furthermore, cells of a patient have been exposed to factors related to the disease (e.g. elevated cortisol levels and probably various other as yet unknown factors), which can cause differences in gene expression status of cells between healthy controls and patients. These differences can be visualized by analyzing gene expression such as RNA levels. If required, cells can be contacted with a stimulus for enhancement of differences in gene expression.

Thus, polymorphic genes and the internal and external (with or without stimulus) cell environment provide a unique combination resulting in differences in gene expression patterns between patients and controls.

A preferred method according to the invention further comprises providing an RNA sample from said cell isolate after contacting said cell isolate with a stimulus, Said stimulus can be selected from cytokines such as stem cell factor, colony-stimulating factor, hepatocyte growth factor, interferon, leukemia inhibitory factor, transforming growth factor beta, tumor necrosis factor alpha, and interleukins; lipopolysaccharides (LPS); neurotransmitters such as acetylcholine, norepinephrine, dopamine, serotonin, and gamma aminobutyric acid; and hormones such as vasopressin, thyroid hormone, oestradiol, progesteron, testosteron, glucocorticoid and dehydroepiandosteron. Preferred stimuli are interleukin (IL) 6 and IL 10, TNF alpha; glucocorticoid, and LPS. A particularly preferred stimulus is provided by LPS, which appeared to be a potent stimulus especially for blood cells.

Preferably, said typing in a method according to the invention is based on a comparison with a reference. In one embodiment, said reference comprises the corresponding RNA levels for said set of genes in an RNA sample from a cell isolate from an individual or a group of individuals not suffering and/or not at risk of suffering from said psychiatric disorder. Alternatively, said reference comprises the corresponding RNA levels for said set of genes in an RNA sample from a cell isolate from an individual or a group of individuals suffering from said psychiatric disorder. In a preferred embodiment, said reference sample comprises both samples from an individual or a group of individuals not suffering and/or not at risk of suffering from said psychiatric disorder and from an individual or a group of individuals suffering from said psychiatric disorder. It is preferred that said group of individuals comprises at least two individuals, more preferred at least three individuals, more preferred at least four individuals, more preferred at least five individuals, more preferred at least ten individuals. A preferred reference comprises RNA levels determined for said set of genes in a cell isolate prior to and/or after contacting said cell isolate with a stimulus.

In a preferred embodiment, said RNA levels from a reference is stored on an electronic storage device, such as, but not limited to, a computer or a server. Said reference can be addressed to compare the determined RNA levels of an individual suffering from a psychiatric disorder, or at risk of suffering there from, with said reference. Said comparison preferably provides a resemblance score, which is a measure for the similarity of the determined RNA levels of said individual with the corresponding levels in said reference. An arbitrarily determined threshold can be provided to classify an individual into a group with a high resemblance score as compared to said reference, and a group with a low resemblance score as compared to said reference.

The invention further provides a method for typing a cell isolate of an individual suffering from a psychiatric disorder, or suspected of suffering there from, the method comprising providing a first RNA sample from a cell isolate from said individual, providing a second RNA sample from said cell isolate after contacting said cell isolate with a stimulus, determining RNA levels for a set of genes in said first and second RNA sample, wherein said set of genes comprises at least two of the genes having the gene number 1-106 listed in Table 9 (first column), more preferably at least two of the genes having the gene number 1-142 listed in Table 9 (first column), and typing said isolate on the basis of the levels of RNA determined for said set of genes.

Preferably, said typing is based on the levels of RNA as determined in said RNA sample provided after contacting the cell isolate with a stimulus, or whereby said typing is based on the ratio of RNA levels determined in said RNA samples provided from a cell isolate prior to an after contacting with a stimulus. It was found by the inventors that typing based on levels of RNA determined for said set of genes after contacting cells with a stimulus, or based on the ratio of RNA levels before and after contacting said cell isolate with a stimulus, provides a more robust typing whereby more information about the disease state of the individual is captured.

A psychiatric disorder comprises a clinical disorder, a mental disorder, a psychotic disorder such as schizophrenia, a depressive disorder, a substance-related disorder such as an alcohol-related disorder or a sedative-related disorder, a somatoform disorder, a factitious disorder, a dissociative disorder, or a personality disorder. A preferred psychiatric disorder comprises a depressive disorder.

The term cell isolate refers to a tissue sample comprising cells from an individual. Preferably, said tissue sample is a sample that can easily be isolated from said individual, including but not limited to a biopsy such as for example a skin biopsy comprising keratinocytes, a buccal swap comprising mucosa cells, and blood comprising blood cells. A preferred tissue sample for use in a method of the invention is provided by whole blood comprising blood cells such as peripheral mononuclear blood cells (PBMC). If required, PBMC can be isolated from an individual in sufficient quantifies, allowing typing of said cells according to a method of the invention. The cells from said tissue sample can be dissociated, if required, to obtain a preferably single cell suspension. Methods for dissociation of cells, employing for example proteases such as collagenase and trypsin, are known by a skilled artisan. An RNA sample can be obtained from said cell isolate by known methods including, for example, Trizol (Invitrogen; Carlsbad, Calif.), RNAqueous® Technology (Qiagen; Venlo, the Netherlands), Maxwell™ 16 Total RNA Purification Kit (Promega; Madison, Wis.) and, preferably, PAXgene™ Blood RNA Kit (Qiagen; Venlo, the Netherlands).

A particularly preferred tissue sample is whole blood comprising blood cells.

If required, said cell isolate can be stored prior to providing a RNA sample from said cell isolated, under conditions that preserve the quality of the RNA. Examples of such preservative conditions are known in the art and include fixation using e.g. formaline, the use of RNase inhibitors such as RNAsin™ (Pharmingen) or RNAsecure™ (Ambion), the use of preservative solutions such as RNAlater™ (Ambion), and reagents such as guananidine thiocyanate, or comparable reagents, for example, as used in the PAXgene™ Blood RNA Kit.

A cell isolate can be contacted with a stimulus by incubating said cell isolate, preferably comprising single cells, with said stimulus. For this, said cell isolate can be incubated or cultured in a medium suited for said cell isolate under conditions that favor survival of the cells. Said contacting can be for a predetermined period of time, ranging from between about 1 minute to several days. A preferred time period ranges between about 1 hour and about 24 hours, more preferred between about 2 hours and about 12 hours, and more preferred is about 5 hours. Methods and means for incubating or culturing a cell isolate are known in the art and can be obtained from, for example, Sigma-Aldrich and Invitrogen-Gibco.

Methods for determining RNA levels for a set of genes are known in the art and comprise Northern blotting, amplification methods such as based on polymerase chain reaction (PCR) and nucleic acid sequence based amplification (NASBA), and array-based methods. Preferred PCR-based methods comprise multiplex PCR and multiplex ligation-dependent probe amplification (Eldering et al., (2003) Nucleic Acids Res. 31: e153). A more preferred method is real-time quantitative PCR (Bustin (2002) J Mol Endocrin 29: 23-39).

An array format is particularly useful for this purpose. An array comprises probes specific for a gene or gene product in an arrayed format on a solid support. Said probes comprise nucleic acid molecules or mimics thereof such as peptide nucleic acid (PNA) that can hybridize to a labeled copy of RNA isolated from a cell isolate. Said probes preferably comprise a stretch of at least 20 nucleic acid residues that are identical to, or at least 95% similar to, a stretch of nucleic acid residues on a RNA molecule of which the level is to be determined, allowing base-pairing between said probe and said RNA molecule or a labeled copy thereof.

Methods for direct of indirect labeling RNA are known in the art and include Fluorescent Direct Label Kit (Agilent Technologies), GeneBeam™ First Strand cDNA Labeling kit (Enzo Life Sciences), and SuperScript™ Direct or Indirect cDNA Labeling Module (Invitrogen). The label preferably comprises fluorescent label such as cyanine 3 and cyanine 5. In a preferred embodiment, said first and second RNA sample are labeled with different dyes, allowing simultaneous hybridization of the labeled samples to a single array. Suitable hybridization and washing conditions as described in, for example, protocols from Agilent and Affymetrix can be applied for hybridization and washing of the arrays. A confocal scanning device is used to determine the intensity of label that remained associated with a probe, as a measure for the level of RNA present in a cell isolate. Different bioinformatic software tools, such as, for example, Agilent Feature Extraction, Limma®, Edwards, Loess, and Aquantile, can be applied to analyze the data. Data analysis comprises normalization of the data to reduce bias within and between experiments, such as dye switch and dye swap (see, for example, Sterrenburg et al., Nucleic Acids Res. 2002 Nov. 1; 30(21):e116).

A depressive disorder refers to a disorder causing consistent loss of interest or pleasure in daily activities for at least a 2 week period, and affecting social, occupational, educational or other important functioning. Said depressive disorder can be dysthymia, a bipolar disorder or manic-depressive illness, or, preferably, major depression which is also known as clinical depression, unipolar depression, and major depressive disorder (MDD). A method of the invention can also be applied for typing a cell isolate of an individual suffering from: 1) MDD or subtypes of MDD comprising melancholic depression, atypical depression, double depression, and MDD with anger attacks; or 2) anxiety disorders (i.e. panic disorder, generalized anxiety disorder, phobia, post-traumatic stress disorder, obsessive-compulsive disorder,

The genes listed in Table 9 with gene numbers 1-106 (first column) were identified because probes specific for these genes yielded a low p-value (t-test for MDD versus Control, <0.005), a low false discovery rate (FDR) of less than 0.01%, and yielded a large effect size (>30%, M-data; >30% R-data. The FDR of a set of predictions is the expected percentage of false predictions in the set of predictions.

The genes listed in Table 9 with gene number numbers 1-142 (which have in addition the alternative numbers 1-142 as present in the second column of Table 9) were identified because probes specific for these genes yielded a good prediction for the disease, and the initial set of 270 was further selected for optimal performance: high participation (100% in PAM-score), high difference between expression values from MDD patients and control (e.g. 0.3≦|Difference_{MDD vs. control})|<0.4=2 points, 0.4≦|Difference_{MDD vs. control)}|<0.5=3 points), low p-value in t-test for MDD vs. Control (P<0.02; e.g. 0.01<P-value≦0.02=2 points, 0.007<P-value≦0.01=3 points), low q-score for MDD vs. Control in SAM analysis (25<q-value<40=3 points, 0<q-value<25=4 points, and when a gene was represented by multiple probes, the replicates should have similar values. Genes with a score>15 were selected for the MDD-marker.

It has been found that the inclusion at least two genes numbered 1-106 (first column) of Table 9 in a method of the invention already provides a good prediction of the sample as being derived from an individual suffering from depression or not. A more accurate prediction is possible by including more genes numbered 1-106 (first column) of Table 9 in a method of the invention.

It has been found that the inclusion at least two genes numbered 1-142 (first column) of Table 9 in a method of the invention also provides a good prediction of the sample as being derived from an individual suffering from depression or not. A more accurate prediction is possible by including more genes numbered 1-142 (first column) of Table 9 in a method of the invention.

Preferably at least three of the genes numbered 1-106 (first column) in Table 9, more preferred at least four of the genes numbered 1-106 (first column) in Table 9, more preferred at least five of the genes numbered 1-106 (first column) in Table 9, more preferred at least six of the genes numbered 1-106 (first column) in Table 9, more preferred at least seven of the genes numbered 1-106 (first column) in Table 9, more preferred at least eight of the genes numbered 1-106 (first column) in Table 9, more preferred at least nine of the genes numbered 1-106 (first column) in Table 9, more preferred at least ten of the genes numbered 1-106 (first column) in Table 9, more preferred at least eleven of the genes numbered 1-106 (first column) in Table 9, more preferred at least twelve of the genes numbered 1-106 (first column) in Table 9, more preferred at least thirteen of the genes numbered 1-106 (first column) in Table 9, more preferred at least fourteen of the genes numbered 1-106 (first column) in Table 9, more preferred at least fifteen of the genes numbered 1-106 (first column) in Table 9, more preferred at least sixteen of the genes numbered 1-106 (first column) in Table 9, more preferred at least seventeen of the genes numbered 1-106 (first column) in Table 9, more preferred at least eighteen of the genes numbered 1-106 (first column) in Table 9, more preferred at least nineteen of the genes numbered 1-106 (first column) in Table 9, more preferred at least twenty of the genes numbered 1-106 (first column) in Table 9, more preferred at least twenty-one of the genes numbered 1-106 (first column) in Table 9, more preferred at least twenty-two of the genes numbered 1-106 (first column) in Table 9, more preferred at least thirty of the genes numbered 1-106 (first column) in Table 9, more preferred at least fifty of the genes numbered 1-106 (first column) in Table 9, most preferred all of the genes numbered 1-106 (first column) in Table 9 are included in a method of the invention.

In another embodiment, is a method including at least two of the genes (numbered 1-142, first column) listed in Table 9, or analogs thereof. Analogs, which include splice variants of said genes are listed in Table 9 as an alternative Unigene cluster or as an alternative Genebank ID. More preferred is a method including three of the genes listed in Table 9, more preferred at least four of the genes listed in Table 9, more preferred at least five of the genes listed in Table 9, more preferred at least six of the genes listed in Table 9, more preferred at least seven of the genes listed in Table 9, more preferred at least eight of the genes listed in Table 9, more preferred at least nine of the genes listed in Table 9, more preferred at least ten of the genes listed in Table 9, more preferred at least eleven of the genes listed in Table 9, more preferred at least twelve of the genes listed in Table 9, more preferred at least thirteen of the genes listed in Table 9, more preferred at least fourteen of the genes listed in Table 9, more preferred at least fifteen of the genes listed in Table 9, more preferred at least sixteen of the genes listed in Table 9, more preferred at least seventeen of the genes listed in Table 9, more preferred at least eighteen of the genes listed in Table 9, more preferred at least nineteen of the genes listed in Table 9, more preferred at least twenty of the genes listed in Table 9, more preferred at least twenty-one of the genes listed in Table 9, more preferred at least twenty-two of the genes listed in Table 9, more preferred at least thirty of the genes listed in Table 9, more preferred at least fifty of the genes listed in Table 9, most preferred all of the genes listed in Table 9 are included in a method of the invention.

In a preferred embodiment, a method according the invention comprises at least two of the genes numbered 1-106 (first column) in Table 9, whereby said genes are numbered 1 and 2. An even more preferred method according to the invention comprises the genes numbered 1-6 of the genes numbered 1-106 (first column) in Table 9, more preferred the genes numbered 1-8 of the genes numbered 1-106 (first column) in Table 9, more preferred the genes numbered 1-12 of the genes numbered 1-106 (first column) in Table 9, more preferred the genes numbered 1-13 of the genes numbered 1-106 (first column) in Table 9 (HBG1, KRT23, AL833005, Caprin1, CENTD3, PROK2, ZBTB16, F11R, FANCE, LOC150166, TMEM4, SLC7A7, and MLC1), more preferred the genes numbered 1-22 of the genes numbered 1-106 (first column) in Table 9.

In another preferred embodiment, a method according the invention comprises at least two of the genes listed in Table 9, whereby said genes have the alternative gene numbers (second column) 1 and 2. In another preferred embodiment, a method according to the invention comprises at least two of the genes listed in Table 9, whereby said genes are CAPRIN1 and ZBTB16. An even more preferred method according to the invention comprises the genes having the alternative numbers (second column) 1-7 of the genes listed in Table 9, more preferred the genes having the alternative numbers (second column) 1-8 of the genes listed in Table 9, more preferred the genes having the alternative numbers (second column) 1-12 in Table 9, more preferred the genes having the alternative numbers (second column) 1-13 of the genes listed in Table 9, more preferred the genes having the alternative numbers (second column) 1-22 of the genes listed in Table 9.

In another preferred embodiment, a method according to the invention comprises at least the genes CLEC4A, F11R, TMEM4 and SLC7A7. In another preferred embodiment, a method according to the invention comprises at least the genes CLEC4A, PLSCR1, PROK2, ZBTB16 and MLC1. In another preferred embodiment, a method according to the invention comprises at least the genes CORO1A, FCN2, KRT23, MLC1, NRGN, PLXNB2, PPBP, PTGS1, RNPC1, SOX4 and VAMP8. In another preferred embodiment, a method according to the invention comprises at least the genes MLC1, RNPC1, PROK2, CLEC4A, CAPRIN1 and ZBTB16.

In another preferred embodiment, a method according to the invention further comprises the gene PBPP. In a preferred embodiment a set of genes of the present invention further comprises the gene PBPP.

Typing of a cell isolate of an individual exhibiting symptoms of depression with a method of the invention can be used to determine whether said individual is indeed suffering from a depressive disorder or is suffering from depressive symptoms that will not turn into a depressive disorder. A method of the invention can also be used to determine the biological severity of the depressive disorder. In a preferred embodiment, said typing allows prognosticating the severity of the syndrome, and/or prognosticating a response to medical treatment.

Medical treatment comprises antidepressant medication such as lithium, tricyclic antidepressants (TCAs), monoamine oxidase inhibitor (MAOIs), and selective serotonin reuptake inhibitors such as citalopram (Celexa), fluoxetine (Prozac), paroxetine (Paxil), and sertraline (Zoloft). A method of the invention is preferably used to prognosticate a response of an individual towards treatment with a selective serotonin reuptake inhibitor.

The invention further provides a set of probes for typing a cell isolate of an individual suffering from a depressive disorder or suspected of suffering there from, whereby said set of probes comprises nucleic acid sequences specific for at least two of the genes numbered 1-106 (first column) in Table 9. In another preferred embodiment, said set of probes comprises nucleic acid sequences specific for at least two of the genes listed in Table 9. Said set of probes preferably comprises at least two probes specific for genes numbered 1-106 (first column) in Table 9, wherein each of said at least two probes is specific for a different gene of the genes numbered 1-106 (first column) of Table 9. In another preferred embodiment, said set of probes preferably comprises at least two probes specific for genes listed in Table 9, wherein each of said at least two probes is specific for a different gene of Table 9. Preferably said set of probes comprises at least three probes that are specific for different genes of the genes numbered 1-106 (first column) in Table 9. In another preferred embodiment, said set of probes comprises at least three probes that are specific for different genes listed in Table 9. Said set of probes preferably comprises probes that are specific for each of the genes numbered 1-106 (first column) in Table 9. In another preferred embodiment, said set of probes preferably comprises probes that are specific for each of the genes listed in Table 9. Said probes are preferably selected to hybridize to specific exons of the genes numbered 1-106 (first column) in Table 9, or to hybridize to the 3′ ends of messenger RNA corresponding to at least two of the genes numbered 1-106 (first column) in Table 9. In another preferred embodiment, said probes are preferably selected to hybridize to specific exons of the genes listed in Table 9, or to hybridize to the 3′ ends of messenger RNA corresponding to at least two of the genes listed in Table 9. Methods for designing said probes specific for the genes numbered 1-106 (first column) or for said probes specific for the genes listed in Table 9 are known in the art and have been discussed, for example, in Bouwman et al. (2006) J Neurochem 99: 84-96; and Stam et al. (2007) Eur. J. Neurosci: In press).

In a preferred embodiment, said set of probes is capable of hybridizing to at least two of the genes numbered 1-106 (first column) in Table 9, and/or an RNA product thereof. In a more preferred embodiment, said set of probes is capable of hybridizing to at least two of the genes listed in Table 9, and/or an RNA product thereof. Said set of probes preferably comprises between 2 and 500 different probes, wherein at least two of said probes are specific for a different gene numbered 1-106 (first column) in Table 9, or more preferably for any different gene listed in Table 9. Preferably said set comprises between 10 and 100 different probes wherein at least two of said probes are specific for a different gene numbered 1-106 (first column) in Table 9, or more preferably for any different gene listed in Table 9. In a particularly preferred embodiment said set comprises 20 probes, wherein each of said probes is specific for a different gene numbered 1-106 (first column) in Table 9, or more preferably for any different gene listed in Table 9.

In another preferred embodiment, a method according to the invention said set of probes comprises at least probes specific for the genes CLEC4A, F11R, TMEM4 and SLC7A7. In another preferred embodiment, a method according to the invention, said set of probes comprises at least probes specific for the genes CLEC4A, PLSCR1, PROK2, ZBTB16 and MLC1. In another preferred embodiment, a method according to invention said set of probes comprises at least probes specific for the genes CORO1A, FCN², KRT23, MLC1, NRGN, PLXNB2, PPBP, PTGS1, RNPC1, SOX4 and VAMP8. In another preferred embodiment, a method according to the invention said set of probes comprises at least probes specific for the genes MLC1, RNPC1, PROK2, CLEC4A, CAPRIN1 and ZBTB16.

In another preferred embodiment, a set of probes according to the invention further comprises a probe specific for the gene PBPP.

In further preferred embodiment, said set of probes comprises 12 probes, wherein said set of probes is specific for the genes numbered 1-12 of the genes numbered 1-106 (first column) in Table 9. The RNA levels of the genes numbered 1-12 of the genes numbered 1-106 (first column) in Table 9, HBG1, KRT23, AL833005, Caprin1, CENTD3, PROK2, ZBTB16, F11R, FANCE, LOC150166, TMEM4, SLC7A7, are preferably used for typing a cell isolate of an individual suffering from a depressive disorder.

In another further preferred embodiment, said set of probes comprises 7 probes, wherein said set of probes is specific for the genes having the alternative numbers (second column) 1-13 in Table 9. The RNA levels of the genes having the alternative numbers (second column) 1-13 in Table 9, KRT23, CAPRIN1, PLSCR1, PROK2, ZBTB16, TMEM4, CLEC4A, MLC1, are preferably used for typing a cell isolate of an individual suffering from a depressive disorder.

In another preferred embodiment, a method according to the invention further comprises the use of RNA levels of the gene PBPP for typing a cell isolate of an individual suffering from a depressive disorder.

In another preferred embodiment, said set of probes comprises no more than 1000 probes, preferably no more than 900, preferably no more than 800, preferably no more than 700, preferably no more than 600, preferably no more than 500, preferably no more than 400, preferably no more than 300, preferably no more than 200, preferably no more than 142, preferably no more than 125, preferably no more than 106, preferably no more than 90, preferably no more than 80, preferably no more than 70, preferably no more than 60, preferably no more than 50, preferably no more than 40, preferably no more than 30, preferably no more than 25, preferably no more than 20, preferably no more than 15, preferably no more than 10.

The invention also provides the use of the set of probes according to the invention for prognosticating syndrome severity, and/or a response to medical treatment for an individual suffering from a depressive disorder.

In another embodiment, the invention provides a set of primers for typing a cell isolate of an individual suffering from a depressive disorder or suspected of suffering there from, whereby said set of primers comprises primers specific for at least two of the genes numbered 1-106 (first column) of Table 9 In another embodiment, the invention provides a set of primers for typing a cell isolate of an individual suffering from a depressive disorder or suspected of suffering there from, whereby said set of primers comprises primers specific for at least two of any of the genes listed in Table 9. It is preferred that primers specific for a gene numbered 1-106 (first column) or any gene of Table 9 can be used in an amplification method to determine a level of expression of the said gene numbered 1-106 (first column) or any gene of Table 9, Therefore, it is preferred that these primer result in the amplification of not more than 2 kilobases of a continuous stretch of nucleic acid sequences on a mRNA product of said gene numbered 1-106 (first column) or any gene of Table 9, more preferred not more that 1 kilobase, most preferred between 50 bases and 200 bases. It is furthermore preferred that said stretch of nucleic acid sequences on a mRNA product span an exon-intron boundary in said gene numbered 1-106 (first column) or any gene of Table 9.

In another preferred embodiment, a method according to the invention said set of primers comprises at least primers specific for the genes CLEC4A, F11R, TMEM4 and SLC7A7. In another preferred embodiment, a method according to the invention, said set of primers comprises at least primers specific for the genes CLEC4A, PLSCR1, PROK2, ZBTB16 and MLC1. In another preferred embodiment, a method according to invention said set of primers comprises at least primers specific for the genes CORO1A, FCN2, KRT23, MLC1, NRGN, PLXNB2, PPBP, PTGS1, RNPC1, SOX4 and VAMP8. In another preferred embodiment, a method according to the invention said set of primers comprises at least primers specific for the genes MLC1, RNPC1, PROK2, CLEC4A, CAPRIN1 and ZBTB16.

In another preferred embodiment, said set of primers comprises no more than 1000 primers, preferably no more than 900, preferably no more than 800, preferably no more than 700, preferably no more than 600, preferably no more than 500, preferably no more than 400, preferably no more than 300, preferably no more than 200, preferably no more than 142, preferably no more than 125, preferably no more than 100, preferably no more than 90, preferably no more than 80, preferably no more than 70, preferably no more than 60, preferably no more than 50, preferably no more than 40, preferably no more than 30, preferably no more than 25, preferably no more than 20, preferably no more than 15, preferably no more than 10.

The invention furthermore provides the use of the set of primers according to the invention for prognosticating syndrome severity, and/or a response to medical treatment for an individual suffering from a depressive disorder.

### DESCRIPTION OF THE FIGURES

FIG. 1. LPS-stimulation reveals significant differences between MDD patients and controls. A false-discovery rate (FDR) analysis was used to detect differentially expressed genes discriminative for disease state in the total group of 33 MDD patients and 34 healthy controls used on the microarray. For each data set (basal: A; LPS: B) the FDR % is plotted to the total number of genes found. The dotted line indicates the 5% FDR limit.

FIG. 2. Confirmation of differential expression level in MDD patients and controls of classifier genes. From 8 genes for which specific qPCR primers could be designed, 7 genes showed a similar difference in expression levels between MDD patients and controls (loge-scale) of the training set using microarray and real-time qPCR. These genes were further used to determine the MDD-marker score. Data are average expression values±s.e.m.

FIG. 3. Molecular marker for MDD. The marker scores were determined for the training (left) and validation set (right) of MDD patients and healthy controls using microarray and real-time qPCR. For both sets and both methods, MDD patients were significantly different from controls (for p-values, see Tables 2 and 3). Average MDD score±s.e.m. are shown.

FIG. 4. Validation of MDD-marker by qPCR. (A) Correlation of MDD-marker (7 genes) using microarray and qPCR. A good correlation (Spearman's rho 0.489, p<0.001; black line) was obtained for the MDD-marker from MDD patients and healthy control as measured by microarray and qPCR. The molecular marker as measured using qPCR in the training set (B) and validation set (C) showed negative values for healthy controls (open square) and positive scores for MDD patients. In both sets MDD patients were significantly different from controls as determined by different statistical tests (Table 3).

FIG. 5. LPS-induced gene expression measured in whole blood samples. Histogram of LPS-induced regulation ([LPS minus basal expression values]; r) of genes detected by FDR (<0.0001) using the expression levels of all cases (67 cases) analyzed. Using stringent criteria (−0.8>r>0.8), about 2100 probes (8%), corresponding to 1725 genes showed a significant regulation, with 65% of these up-regulated and 35% down-regulated. Regulation bins and an arbitrarily chosen cut-off for regulation (gray line, are indicated. For typical LPS-induced genes, see Table 6.

FIG. 6. Overrepresentation analysis of MDD marker genes by Gene Ontology (GO) class Biological process. From the 7 MDD marker genes, 5 genes were assigned a GO function for biological process. For each level, a number indicates which gene participates in the subclass. Subclasses in red are overrepresented compared with the genes from the total microarray (present genes only).

FIG. 7. LPS-induced gene expression measured in whole blood. Histogram of LPS-induced regulation ([LPS-basal]; r) of genes detected by FDR (<0.0001) using the expression levels of all cases (67 persons) analyzed. About 1700 probes (7% of total) showed a significant regulation, with 65% of these up-regulated and 35% down-regulated. Regulation bins and an arbitrarily chosen cut-off for regulation (gray line, −0.8>r>0.8) are indicated.

FIG. 8. LPS-stimulation reveals significant differences between MDD patients and controls. A false-discovery rate (FDR) analysis was used to detect differentially expressed genes discriminative for disease state in the total group of 33 MDD patients and 34 healthy controls. For each data set (basal (A), LPS (B), and [LPS-basal] (C)) the FDR % is plotted to the total number of genes found. In particular, the [LPS-basal] data contained several significant differentially expressed genes at FDR≦5% and <10% (27 and 126 genes, respectively). Note the difference in scale. See FIG. 9 for clustering.

FIG. 9. Hierarchical clustering segregates MDD patients from healthy controls. Genes (126) that were differentially expressed between MDD patients (n=33) and healthy controls (n=34) (FDR<0.1) for the [LPS-basal] data were used in complete hierarchical clustering for sample type (MDD vs. control) and genes. This resulted in a separation (two main branches) based on disease status with 72% and 73% of the samples correct (controls, MDD, respectivey; cf. Table 8). Cyan and magenta indicate relative level of normalized gene expression (cyan: induced by LPS; magenta: repressed by LPS) on a loge-scale (see colored bar).

FIG. 10. Differentially expressed genes measured by microarray and real-time qPCR. The expression of four differentially expressed genes (CLEC4A, F11R, TMEM4 and SLC7A7) as measured by microarray in the complete set of 67 subjects was analyzed in an overlapping set of patients and controls using real-time qPCR for the [LPS-basal] data. * p<0.05; # p=0.212. Data are mean expression values±s.e.m.

FIG. 11. Confirmation of expression levels of classifier genes. Classifier genes specific for the data sets from basal and LPS-stimulated samples (A), and for [LPS-basal] (B) were analyzed by real-time qPCR in an overlapping set of patients and controls (MLC1, KRT23, RNPC1, PROK2, CLEC4A, CAPRIN1, ZBTB16). Comparable expression levels and regulation values were obtained as in the microarray data for the complete set of 67 subjects. Cf. FIG. 10 for the three other classifier genes for the [LPS-basal] data (F11R, TMEM4 and SLC7A7). Data are mean expression values±s.e.m.

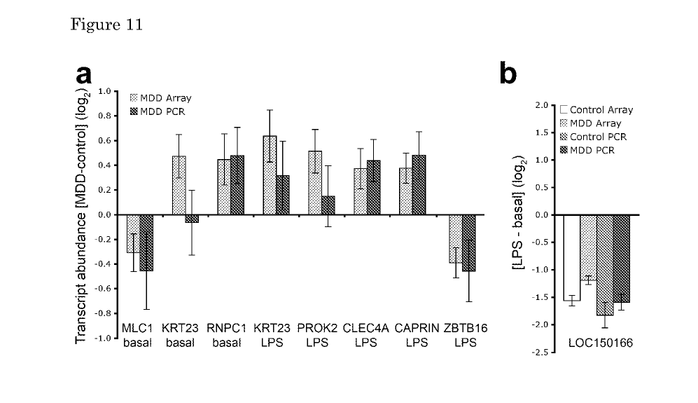

FIG. 12. Scatterplots of False Discovery Rates (FDR %) to the number of genes detected for the ratio (blue), the LPS-induced signal (red) and the baseline signal (green).

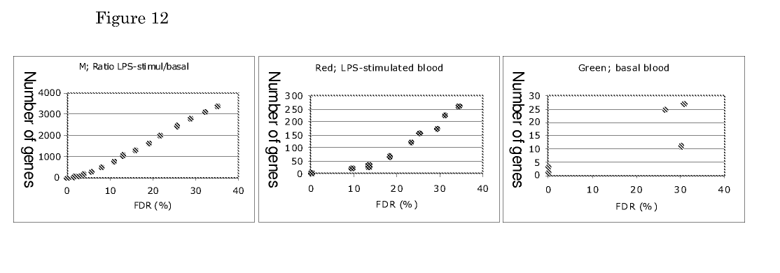

FIG. 13. Pearson correlations to fit the MDD or control profile for 12 classifier genes. When the correlation is positive the profile of an individual resembles that of the mean MDD profile, when negative it fits with the mean control profile. These profiles were as yet generated for 20 MDD patients and 21 healthy controls displayed in the order of the Pearson correlation. Pearson correlations for 5 genes of the LPS-regulated ratio (corrected for base-line; A using microarrays, for 7 genes of the LPS-signal (B), and a summation of Pearson correlation generated by the basal signal for 2 genes, the LPS-signal (7 genes), and the LPS-regulated ratio (5 genes) (C).

FIG. 14. Pearson correlations to fit the MDD or control profile for 20 12 classifier genes of the R-data set. When the correlation is positive the profile of an individual resembles that of the mean MDD profile, when negative it fits with the mean control profile. These profiles were as yet generated for 21 MDD patients and 21 healthy controls displayed in the order of the Pearson correlation. A) Pearson correlations were generated for 12 genes of the LPS-signal using microarrays, and B) for the set of 7 genes of which the differential expression between MDD patients and healthy controls was corroborated by real-time quantitative PCR. Using both methods, these genes could distinguish MDD patient and healthy controls significantly (P=1.5. 10⁻⁵ (A), P=5.7. 10⁻³ (B)).

### REFERENCES

Artigas F, Romero L, de Montigny C, Blier P (1996). Acceleration of the effect of selected antidepressant drugs in major depression by 5-HT1A antagonists. Trends Neurosci 19(9): 378-83.
Azmitia E C, Segal M (1978). An autoradiographic analysis of the differential ascending projections of the dorsal and median raphe nuclei in the rat. J Comp Neurol 179: 641-668.
Blier P, Bergeron R (1998). The use of pindolol to potentiate antidepressant meditatiën. J Clin Psychiatry 59 Suppl 5: 16-23; discussion 24-5.
Bouwman J, Spijker S, Schut D, Wächtler B, Ylstra B, Smit A B, Verhage M. Reduced expression of neuropeptide genes in a genome-wide screen of a secretion-deficient mouse. J. Neurochem. 2006 October; 99(1):84-96.
Caspi A, Sugden K, Moffitt T E, Taylor A, Craig I W, Harrington H, McClay J, Mill J, Martin J, Braithwaite A, Poulton R (2003). Influence of life stress on depression: moderation by a polymorphism in the 5HTT gene. Science 301: 386-9.
Celada P, Puig M V, Casanovas J M, Guillazo G, Artigas F (2001). Control of dorsal raphe serotonergic neurons by the medial prefrontal cortex: Involvement of serotonin-1A, GABA(A), and glutamate receptors J Neurosci 21(24): 9917-29.
Drevets W C (2000). Neuroimaging studies of mood disorders. Biol Psychiatry 48(8): 813-29.
Drevets W C, Price J L, Simpson J R, Todd R D, Reich T, Vannier M, Raichle M E (1997). Subgenual prefrontal cortex abnormalities in mood disorders. Nature 386: 824-827.
Flint J, Corley R, DeFries J C, Fulker D W, Gray J A, Miller S, Collins A C (1995). A simple genetic basis for a complex psychological trait in laboratory mice. Science 269: 1432-1435.
Fuster J M (1997). In: The prefrontal cortex. Anatomy, physiology and neuropsychology of the frontal lobe. New York: Lippincott-Raven.
Gross C, Zhuang X, Stark K, Ramboz S, Oosting R, Kirby L, Santarelli L, Beck S, Hen R (2002). Serotonin1A receptor acts during development to establish normal anxiety-like behaviour in the adult. Nature 416(6879): 396-400.
Haddjeri N, Blier P, de Montigny C (1998). Long-term antidepressant treatments result in a tonic activation of forebrain 5-HT1A receptors. J Neurosci 18(23): 10150-6.
Hajós M, Richards C D, Szekely A D, Sharp T (1998). An electrophysiological and neuroanatomical study of the medial prefrontal cortical projection to the midbrain raphe nuclei in the rat. Neuroscience 87: 95108.
Hettema J M, Neale M C, Kendler K S. A review and meta-analysis of the genetic epidemiology of anxiety disorders. Am J. Psychiatry. 2001 October; 158(10): 1568-78.
Holsboer F, Lauer C J, Schreiber W, Krieg J C (1995). Altered hypothalamic-pituitary-adrenocortical regulation in healthy subjects at high familial risk for affective disorders. Neuroendocrinology 62: 340-7.
Inder W J, Prickett T C, Mulder R T, Donald R A, Joyce P R (2001). Reduction in basal afternoon plasma ACTH during early treatment of depression with fluoxetine. Psychopharmacology (Berl) 156(1): 73-8.
Jakab R L, Goldman-Rakic PS (2000). Segregation of serotonin 5-HT2A and 5-HT3 receptors in inhibitory circuits of the primate cerebral cortex. J Comp Neurol 417(3): 337-48.
Jorgensen H, Kjaer A, Knigge U, Moller M, Warberg J (2003). Serotonin stimulates hypothalamic mRNA expression and local release of neurohypophysial peptides. J Neuroendocrinol 15(6): 564-71.
Kagamiishi Y, Yamamoto T, Watanabe S (2003). Hippocampal serotonergic system is involved in anxiety like behavior induced by corticotropin-releasing factor. Brain Res 991(1-2): 212-21.
Kia H K, Miguel M C, Brisorgueil M J, Daval G, Riad M, El Mestikawy S, Hamon M, Vergé D (1996). Immunocytochemical localization of serotonin(1A) receptors in the rat central nervous system. J Comp Neurol 365: 289-305.
Kirschbaum C, Hellhammer D H (1989). Salivary cortisol in psychobiological research: an overview. Neuropsychobiology 22(3): 150-69.
Kunzel H E, Zobel A W, Nickel T, Ackl N, Uhr M, Sonntag A, Ising M, Holsboer F (2003). Treatment of depression with the CRH-1-receptor antagonist R121919: endocrine changes and side effects. J Psychiatr Res 37(6): 525-33.
Mann J J, Huang Y Y, Underwood M D, Kassir S A, Oppenheim S, Kelly T M, Dwork A J, Arango V (2000). A serotonin transporter gene promoter polymorphism (5-HTTLPR) and prefrontal cortical binding in major depression and suicide. Arch Gen Psychiatry 57(8): 729-38.
McGuire M T, Troisi A (1998). Prevalence differences in depression among males and females: are there evolutionary explanations? Br J Med Psychol 71: 479-91.
Merikangas K (2002). Genetic epidemiology: bringing genetics to the population—the NAPE lecture 2001. Acta Psych Scand 105: 3-13.
Montgomery A J, Bench C J, Young A H, Hammers A, Gunn R N, Bhagwagar Z, Grasby P M (2001). PET measurement of the influence of corticosteroids on serotonin-1A receptor number. Biol Psychiatry 50(9): 668-76.
Neumeister A, Konstantinidis A, Stastny J, Schwarz M J, Vitouch O, Willeit M, Praschak-Rieder N, Zach J, de Zwaan M, Bondy B, Ackenheil M, Kasper S (2002). Association between serotonin transporter gene promoter polymorphism (5-HTTLPR) and behavioral responses to tryptophan depletion in healthy women with and without family history of depression. Arch Gen Psychiatry 59(7): 613-20.
Oshima A, Flachskamm C, Reul J M, Holsboer F, Linthorst A C (2003). Altered serotonergic neurotransmission but normal hypothalamic-pituitary-adrenocortical axis activity in mice chronically treated with the corticotropin-releasing hormone receptor type 1 antagonist NBI30775. Neuropsychopharmacology 28(12): 2148-59.
Peyron C, Luppi P H, Kitahama K, Fort P, Hermann D M, Jouvet M (1995). Origin of the dopaminergic innervation of the rat dorsal raphe nucleus. NeuroReport 6: 2527-2531.
Pompeiano M, Palacios J M, Mengod G (1994). Distribution of the serotonin 5-HT2 receptor family messenger-RNAs—comparison between 5-HT(2A) and 5-HT(2C) receptors. Mol Brain Res 23: 163178.
Raadsheer F C, van Heerikhuize J J, Lucassen P J, Hoogendijk W J, Tilders F J, Swaab D F (1995). Corticotropin-releasing hormone mRNA levels in the paraventricular nucleus of patients with Alzheimer's disease and depression. Am J Psychiatry 152(9): 1372-6.
Sargent P A, Kjaer K H, Bench C J, Rabiner E A, Messa C, Meyer J, Gunn R N, Grasby P M, Cowen P J (2000). Brain serotonin1A receptor binding measured by positron emission tomography with [11C]WAY-100635: effects of depression and antidepressant treatment. Arch Gen Psychiatry 57(2): 174-80.
Soares J C, Mann J J (1997). The functional neuroanatomy of mood disorders. Psychiatr Res 31(4): 393-432.
Spijker, J, de Graaf, R, Bijl, R V, Beekman, A T F, Ormel, J, Nolen, W A (2002). Duration of major depressive episodes in the general population. Results from the Netherlands Mental Health Survey and Incidence Study (NEMESIS). Br Psychiatry 181: 208-13.
Stahl, S M (2000). Essential psychopharmacology: neuroscientific basis and practical application. Cambridge University Press, Cambridge, UK.
Stam F J, Macgillavry H D, Armstrong N J, de Gunst M C, Zhang Y, van Kesteren R E, Smit A B, Verhaagen J. Identification of candidate transcriptional modulators involved in successful regeneration after nerve injury. Eur J. Neurosci. 2007 June; 25(12):3629-363.
Sterrenburg E, Turk R, Boer J M, van Ommen G B, den Dunnen J T. A common reference for cDNA microarray hybridizations. Nucleic Acids Res. 2002 Nov. 1; 30(21):e116.
Stockmeier C A, Shapiro L A, Dilley G E, Kolli T N, Friedman L, Rajkowska G (1998). Increase in serotonin1A autoreceptors in the midbrain of suicide victims with major depression-postmortem evidence for decreased serotonin activity. J Neurosci 18(18): 7394-401.
Sullivan P F, Neale M C, Kendler K S (2000). The genetic epidemiology of major depression: review and meta-analysis. Am J Psychiatry 157: 1552-1562.
Summers C H, Kampshoff J L, Ronan P J, Lowry C A, Prestbo A A, Korzan W J, Renner K J (2003). Monoaminergic activity in subregions of raphe nuclei elicited by prior stress and the neuropeptide corticotropin-releasing factor. J Neuroendocrinol 15(12): 1122-33.

### EXAMPLES

### Example 1

An unchallenged sample was taken within 1 h after blood has been withdrawn (and minimally 10 min after). For this, a full heparin tube was inverted 5 times prior to opening the tube. Then, 2.5 ml was transferred to a PAXtube (PreAnalytiX GmbH) by decantation or pipeting (large tip opening). The PAXgene tube was inverted at least 10 times, and kept at room temperature for minimally 2 hours. Then, it was put at −20° C.

A challenged sample was made from the remaining blood in the heparin tube (allowing enough oxygen supply). To the remaining blood 1% (V/V) of LPS (final concentration: 10 ng LPS/ml blood) was added, and the tube was inverted 3 times to mix well immediately after addition of the LPS solution. The tube was kept at 37° C. slowly rotating (or otherwise lying flat slowly shaking) for 5-6 h. After this, the tube was inverted 5 times prior to opening the tube. Then, 2.5 ml was transferred to a PAXtube. This PAXgene tube was inverted at least 10 times, and kept at room temperature for minimally 2 h. Then, it was put at −20° C.

RNA isolation proceeds according to the PAX gene protocol (PreAnalytiX GmbH). After elution the RNA concentration was checked. Samples were subsequently precipitated with 0.3 M NaAc and ethanol with the addition of 0.1 μg linear acrylamide (co-precipitant), and stored at −80° C. until use. Labeling of RNA (1 μg) to use in microarray experiments was according to the Agilent protocol. Labeled RNA (1.5 μg Cy3-labeled basal sample; 1.25 μg Cy-5 labeled LPS sample) was hybridized onto the 44 k Human Agilent whole genome arrays according to the protocol. After washing (Agilent protocol), the arrays were quickly dried using acetonitrile. The array was scanned using the Agilent scanner.

Signal intensities were extracted using Agilent Feature Extraction (v8.0), and the data were analyzed using Limma (R), using median signals and median background. Non-uniform signals were flagged prior to analysis. After background subtraction (Edwards, offset 30), within normalization (Loess) and between array normalization (Aquantile) data were exported to SPSS or Excel. Further data selection consisted of the following criteria (in that order), resulting in ˜25,000 genes:

For any flagged red or green signal, the signal was discarded

Signal intensities for any gene should be >6.8 (log 2)

For any gene the signal should be present in >80% of the control and in >80% of the MDD samples

Subsequent analysis were done on the ratio (LPS vs basal; M), the red (LPS; R) or the green (basal; G) signal.

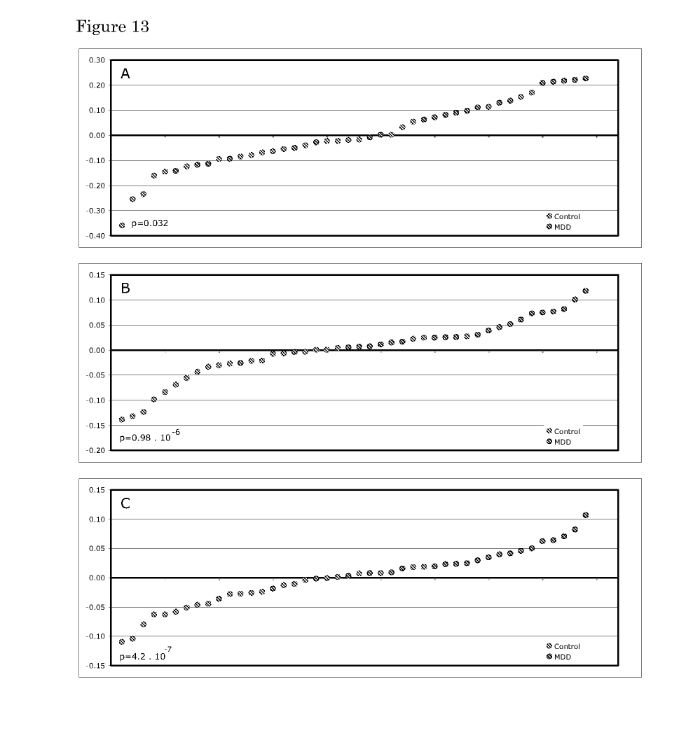

To identify classifier genes, the program PAM was used. Disease-state was used as identifier both for the M-data and the separate R- and G-dataset. The prediction errors were low, i.e. 35-40%. The analysis resulted in sets of 105 probes that participated>90% in the prediction. From these genes a selection was made based on the fact whether probes yielded a low p-value (t-test for MDD versus Control, <0.005 & FDR<0.01%), a large effect size (>30%), and whether multiple probe sets that represent a single gene had low p-values (<0.05) and high effect size (>20%) as well. In addition, the selected probe should be identifiable as an entry in Entrez Gene. As such, an initial selection of 12 genes was made. Some genes participated were informative for both the G- and R-data. For the G- and R-data, as well as for the M-data, the z-translocated Pearson correlation of the expression of each individual to the average of the expression from Controls was subtracted from the Pearson correlation of the expression of each individual to the average of the expression from MDD. Samples with a high score fulfil to the average MDD profile whereas samples with a low score fulfil to the average control profile. Figures representing these correlations for the separate R-data and M-data are shown (FIG. 13A,B, respectively). When the individual scores for the M-data and the G-& R-data were added, an even more robust difference could be made (FIG. 13C).

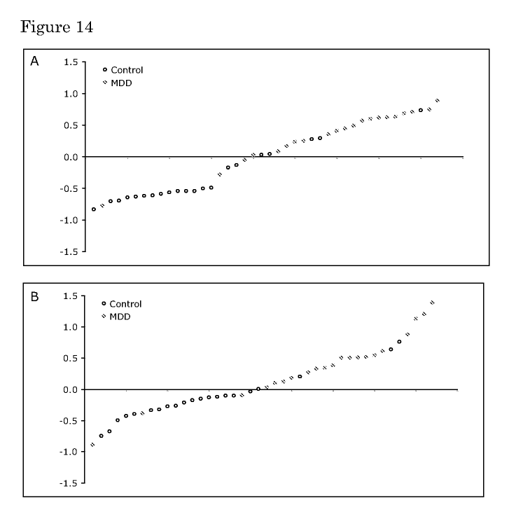

In another case, the program PAM was used to identify classifier genes. Disease-state was used as identifier both for the separate R- and G-dataset. The prediction errors were low, i.e. 35-40%. The PAM analysis resulted in sets of 160, and 110 probes (G- and R-dataset, respectively) that participated>90% in the prediction. From these genes a selection was made based was reduced further by selection based on the effect size (>30% differential expression between MDD and control), p-value (<0.05) and the robustness of the gene in the classifier (classification participation). In addition, the selected probe should be identifiable as an entry in Entrez Gene. As such, an initial selection of 12 genes was made for each data-set. Some genes participated were informative for both the G- and R-data. For the G- and R-data, as well as for the M-data, the z-translocated Pearson correlation of the expression of each individual to the average of the expression from Controls was subtracted from the Pearson correlation of the expression of each individual to the average of the expression from MDD. Samples with a high score fulfil to the average MDD profile whereas samples with a low score fulfil to the average control profile. A figure representing this correlation for the R-data is shown (FIG. 14).

### Example 2

### Introduction

Major Depressive Disorder (MDD) is a highly prevalent psychiatric disorder that accounts for major psychological, physical and social impairments. Life-time prevalence for MDD is estimated from 15-17%, with women being affected twice as often as men^{1,2}. Different factors have been found to play a role in the onset of MDD, including biological³, genetic^{1,4}, and environmental factors (e.g. stress)⁵, but the exact pathogenesis of MDD remains largely unclear. At present, criteria for MDD diagnosis and treatment are based on various signs and symptoms that do not always fit into strict diagnostic categories such as DSM IV. Despite the fact that various risk-factors are known (i.e. genetics, gender, age of onset), biological markers that could support diagnosis, predict the risk for the (re)occurrence of MDD or for treatment outcome are not currently available.

Recent studies have suggested gene-expression profiling in blood cells as a promising alternative for identification of disease classifiers and risk markers⁶⁻⁹. Blood cells could be viewed as biosensors, of which the gene expression is influenced by the surrounding body fluids and all effector molecules therein. As such, gene expression of blood cells might reveal previous or developing disease states and thus present valuable diagnostic markers of an individual, with the perspective of predicting treatment efficacy or a patient's long-term prognosis. As a first step towards this goal, we explored the possibility to segregate subjects with MDD from healthy controls based on gene expression in whole blood. To this end, we examined whole genome gene expression from venous whole blood samples of unmedicated subjects (MDD and healthy controls) from the Netherlands Study of Depression and Anxiety (NESDA) cohort¹⁰.

Generally, a limitation of genomics-based assessment of human samples is individual variability. Despite sample matching, inter-individual and temporal variation in gene expression patterns occur in both brain¹¹ and in whole blood¹². In this study, we applied a powerful gene expression stimulus ex vivo. Previously, we have shown that such a stimulus generates a higher signal to basal state ratio¹³, thereby uniquely revealing differences in genomic responses related to disease state and to the individual's genotype. A lipo-polysaccharide (LPS) stimulus was chosen because it is a strong inducer of gene expression in human monocytes^{13,14}. Moreover, studies on blood lymphocytes have revealed a close association between the state of the immune system and major psychiatric disorders¹⁵⁻¹⁹, and LPS might induce depressive-like behavior when applied in vivo in human subjects as well as in rodents^{20,21}. We used this approach to classify MDD patients based on whole blood gene expression profiles from unmedicated MDD patients and controls selected from the Netherlands Study of Depression and Anxiety and identified a selection of genes of which the expression is predictive for the disease status.

### Methods

### Clinical Study

In this study, we enrolled a total of 35 subjects currently experiencing a single or recurrent MDD episode, and 37 healthy controls with no current or previous diagnosis of MDD (Table 1 and 2); all cases are part of the Netherlands Study of Depression and Anxiety (NESDA; www.nesda.nl) cohort^{10,22}.

For these subjects, blood was taken at two clinical sites (Amsterdam, Leiden). The composite interview diagnostic instrument-lifetime version 2.1-(CIDI²³) was used to diagnose psychiatric disorders according to DSM-IV algorithms²⁴. The Inventory of Depressive Symptomatology-IDS-SR30 was used to define MDD severity¹⁰. MDD cases with IDS scores lower than 21 and healthy controls with scores higher than 13 were excluded from further study. None of the cases and controls had co-morbid physical (e.g. malignancies, cardiovascular, neurological, immune or endocrine disorders) or psychiatric disorders (e.g. personality disorders, threatening compliance and safety) other than the diagnosed MDD and anxiety disorders for the patient group. Nor did any MDD patients or controls have a previous record of taking any medication, with the exception of sporadic use of paracetamol and ibuprofen (i.e. <3 times per week). For all MDD patients, healthy controls were matched for age, sex, smoking (previous, quit or no smoking; and age of onset of smoking), in this order. For females, additional matching criteria consisted of stage of cycle, use of contraceptive drugs, and known pregnancy²⁵. The Medical Ethics committee of the VU University Medical Center approved the study protocol. Written informed consent was obtained from all participants. For a limited set of patients and controls, their leukocyte count was analyzed. There was no difference based on disease status, nor did it correlate with the MDD-marker (Table 4).

### RNA Isolation and Quality Control

Serial venous whole blood samples were obtained between 8:00 a.m. and 10:00 a.m., after overnight fasting, in one 7 ml heparin-coated tube (Greiner). Between 10-60 minutes after blood draw, 2.5 ml of blood was transferred into a PAXgene tube (Qiagen) and used as the basal (non-LPS stimulated) sample. This tube was kept at room temperature for a minimum of 2 h, and subsequently stored at −20° C. The remaining blood (4.5 ml) was stimulated by addition of LPS (10 ng/ml blood; E. coli, Sigma). LPS-stimulated samples were laid flat and incubated at a slow rotation for 5-6 h at 37° C. before a 2.5 ml sample of this LPS-stimulated blood was transferred into a PAXgene tube (Qiagen), and treated as described for the basal sample.

RNA isolation was carried out as described previously¹³ including DNase treatment after thawing of the PAX tubes for 2 h at room temperature. RNA quality was first determined by spectrophotometry (NanoDrop ND-1000 UV-Vis Spectrophotometer, Nanodrop Technologies). Because of contaminants due to the isolation procedure, visible in the spectrophotogram at 200-230 nm, RNA samples were precipitated using ethanol, and NaAc with addition of 0.1 μg linear acryl-amide. Subsequently, RNA quality was determined by spectrophotometry and by using the RNA 6000 NanoChip kit on an Agilent 2100 Bioanalyzer (Agilent Technologies, Santa Clara, Calif.). RNA passing quality control criteria (RIN values>8) were used for further analysis.

### Microarray Procedure

Labeling of RNA (1 μg) was according to the Agilent protocol (Agilent technologies, Palo Alto, USA). Using spectrophotometry, cRNA concentration and labeling efficiency were determined (Nanodrop ND-1000 UV-Vis Spectrophotometer, Nanodrop Technologies). From each patient, labeled cRNA (1.5 μg Cy3-labeled basal blood sample; 1.25 μg Cy-5 labeled LPS-stimulated blood sample) was hybridized for 15-17 h at 65° C. onto the 44K Human Agilent whole genome arrays according to the manufacturer's protocol. After washing, the arrays were quick-dried using acetonitrile. The array was scanned using the Agilent GeneArray Scanner (Agilent G2505B) with default settings for two-color hybridization.

Signal intensities were extracted using Agilent Feature Extraction (v8.0). Data were analyzed with Limma (Bioconductor) using median signals and mean background. Control spots were normalized, but were set not to influence normalization. Non-uniform signals were removed prior to further analysis. After background subtraction (Edwards, offset 30), within normalization (Loess) and between array normalization (Aquantile) data were exported to SPSS. Further data selection consisted of the following criteria (in this order); 1) Signal intensities for any gene should be 1.3× background (>6.8 (log 2)), and have non-saturated levels (<15.7 (log 2)), 2). In addition, for any gene the signal should be present in >80% of the control as well as in >80% of the MDD samples to avoid type-1 errors due to a limited number of participants in each group. These genes were given an absent-call. From the total of 41675 spots, filtering resulted in 22789 spots for the basal sample and 22935 spots in the LPS-stimulated sample, with an overlap of 21107 spots (>92%).

### Micro Array Data Analysis (SAM, PAM)

We used a Significance Analysis for Microarrays (SAM) analysis tool using R functions (Bioconductor, SAMR version 1.2) as an alternative method to identify differentially expressed genes and to estimate a False Discovery Rate (FDR). SAM was carried out on the data set using 100 permutations.

The Prediction Analysis of Microarrays (PAM)²⁶ analysis tool (Bioconductor, pamr version 1.28) was used to generate cross-validated gene lists that distinguish MDD from healthy controls at different misclassification error rates. The randomly assigned first round of 21 MDD patients and 21 matched healthy controls was used as a training set, and the second round of 12 MDD patients and 13 matched healthy controls (of which 2 were not matched on gender) served as a validation set for the microarray. The classification accuracy was assessed by a 10 times repeated 10-fold cross-validation (leave-one-out) on the training set. Genes were further selected for optimal performance: high participation (100% in PAM-score), high difference between expression values from MDD patients and control (e.g. 0.3≦|Difference_{MDD vs. control})|<0.4=2 points, 0.4≦|Difference_{MDD vs. control})|<0.5=3 points), low p-value in t-test for MDD vs. Control (P<0.02; e.g. 0.01<P-value<0.02=2 points, 0.007<P-value<0.01=3 points), low q-score for MDD vs. Control in SAM analysis (25<q-value<40=3 points, 0<q-value<25=4 points, and when a gene was represented by multiple probes, the replicates should have similar values. Genes with a score>15 were selected for the MDD-marker.

The MDD-marker score of an individual sample (i) is defined as its Fischer-z transformed Pearson correlation with the average MDD profile (MDD) minus its Fischer-z transformed Pearson correlation with the average control profile (Control) for the indicated number of genes, based on a calculation by Scherzer et al⁷. The average profile was determined for each set of samples (training and validation set) and for each technique used (microarray and qPCR).

$$MDD - {score}_{i} = 0.5 ln \frac{( 1 + {r}_{{x}_{i} , {\overline{x}}_{MDD}} )}{( 1 - {r}_{{x}_{i} , {\overline{x}}_{MDD}} )} - 0.5 ln \frac{( 1 + {r}_{{x}_{i} , {\overline{x}}_{Control}} )}{( 1 - {r}_{{x}_{i} , {\overline{x}}_{Control}} )}$$

xᵢ={Gᵢ₁, . . . , Gᵢₙ}
Gᵢₙ=gene expression for individual i at gene n
n=number of genes
Real-Time Quantitative PCR (qPCR)

For genes of interest, transcript-specific primers were designed based on Genbank sequence entries using Primer Express software (PE Biosystems, USA) with the manufacturer's settings. Only primers were taken of which the endpoint PCRs showed the amplicon and no primer-dimers as determined by generation of dissociation curves, and which had high amplification efficiencies.

From each sample, random primed (hexamers; Eurogentec, Belgium) cDNA (500 ng total RNA) was made with reverse transcriptase (200 U; Promega, USA) according to the manufacturer's protocol. Aliquots of cDNA were stored at −80° C., because repeated freeze-thaw cycles affect measured Ct values. For qPCR measurements (ABI PRISM 7900, Applied Biosystems), PCR conditions and SYBR green reagents (Applied Biosystems; USA) were used in a reaction volume of 10 μl using transcript-specific primers (300 nM) on cDNA (corresponding to ˜2 ng RNA).

The obtained cycle of threshold (Ctₓ) value for every gene was used to calculate the relative level of gene expression by normalization to the geometric means of replicated reference controls (Ct_{HK}; ACTB, GAPDH, TLN1). For all statistical calculations loge-based values were used for the amount of normalized transcript of interest, C−(Ctₓ−Ct_{HK}) (C=10), or the LPS-induced ratio, −((Ct_{x,LPS}−Ct_{HK, LPS})−(Ct_{x,basal}−Ct_{HK, basal})).

### Statistics

Fischer z-translocated Pearson correlation scores were analyzed by Newman-Keuls, Mann-Whitney U and, and χ² tests. Significance of correlations between IDS and MDD-marker score (two-sided, α=0.05) was tested by Spearman rank correlation coefficient. Significance of individual genes was tested by Newman-Keuls test (two-sided, α=0.05).

### Results

In order to build a molecular marker gene set for MDD based on blood gene expression, we excluded several confounding factors. Because of the large NESDA cohort (1115 MDD patients), we were able to use stringent exclusion criteria to avoid the possibility that medication (e.g. antidepressants or benzodiazepines), or physical disorders could influence gene expression^{27,28}.

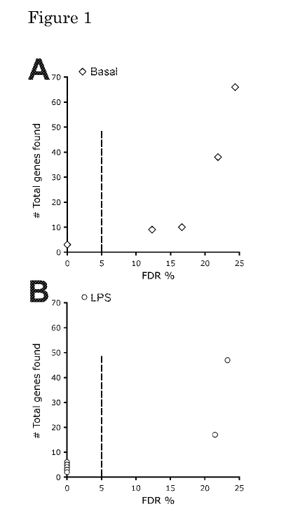

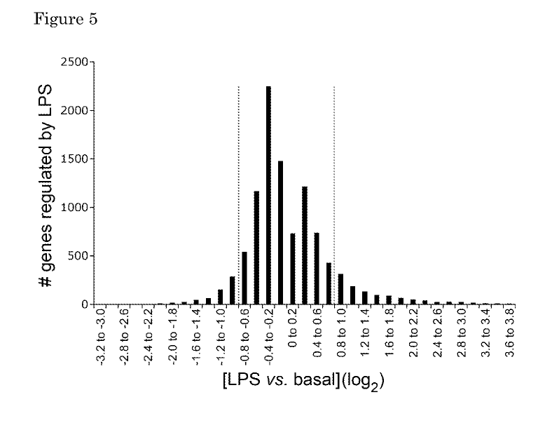

After inclusion of 35 MDD subjects and 37 healthy controls, blood samples from 21 of the MDD patients were randomly taken in a first collection round and were matched with 21 healthy controls (Table 1, Table 5). This first round served as a training set to determine a molecular marker using a classifier approach (see below). In a second round, an additional 12 MDD patients and 13 healthy controls were randomly collected for validation of the molecular MDD-marker by microarray. Microarray analysis with genome-wide coverage was performed for these 67 subjects, and for each subject the labeled cRNA derived from a basal blood sample was co-hybridized with that of an LPS-stimulated blood sample. When the LPS sample was compared with the basal sample for all 67 arrays, about 1700 probes (7%) showed significant regulation according to stringent criteria (FDR<0.0001; −0.8>[LPS vs. basal] (log 2)>0.8), with 65% of these up-regulated and 35% down-regulated (FIG. 5). LPS induced regulation of many known stress-response genes (Table 6). Thus, as we showed previouslyl³, LPS was a powerful stimulus to induce gene expression in peripheral blood. We first analyzed our data for the potential to discriminate for the disease state in order to use it for a later classifier approach (see below); we examined the number of genes that were significantly different in expression levels between MDD patients and healthy controls (43 and 44 subjects, respectively). The differentially-expressed genes that are discriminative for the disease state were analyzed using a false-discovery rate (FDR) analysis. Notably, a low number (3 genes) of significant genes (FDR<0.1) were detected when comparing basal blood samples (FIG. 1a) or blood samples that were stimulated with LPS (6 genes, FIG. 1b).

In order to build a molecular marker set that has diagnostic value for MDD, we used the prediction analysis for microarrays (PAM) tool²⁶ on the training set of 21 MDD patients and 21 healthy controls, to select genes that were tested on an independent validation set of 12 MDD patients and 13 controls. This analysis was performed independently for the basal and the LPS-stimulated sample. In order to use the marker as a diagnostically-applicable tool and to circumvent over-fitting due to large numbers of genes²⁹, the number of classifier genes found using the PAM analysis (160, 110 genes, respectively) was reduced further by selection based on the effect size (differential expression between MDD and control), p-value and the robustness of the gene in the classifier (classification participation). Finally, for each sample, 12 candidate classifier genes were selected (Table 7). The set of classifier genes from the LPS-stimulated sample was slightly better in discriminating MDD patients from controls than the set of classifier genes from the basal sample, as was determined by different statistical tests (Table 2). In order to validate these results, the MDD-marker was evaluated on the independent set of 12 MDD patients and 13 controls. This showed that the classifier genes of the LPS-stimulated sample were superior (p=0.002) to those of the basal sample for discrimination of MDD patients from controls (p=0.311; Table 2).

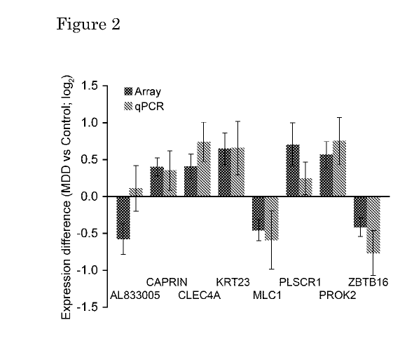

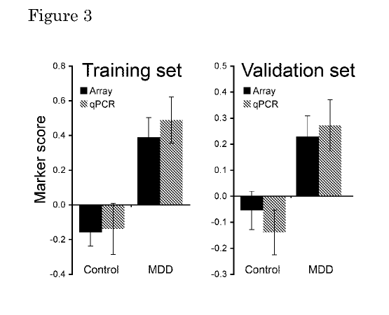

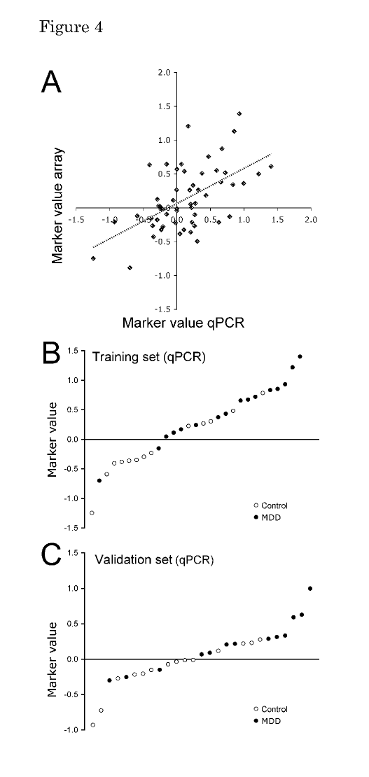

To avoid possible type-1 errors and to reduce the number of classifier genes, we used the technically-independent real-time quantitative PCR (qPCR) technique and analyzed the difference in gene expression in the LPS-stimulated sample in patients and controls of the training set. For 8 out of 12 genes, it was possible to make specific qPCR primers. For 7 out of these 8 genes, the difference in expression level between MDD patients and controls could be corroborated (FIG. 2). This reduced set of 7 classifier genes was still able to separate MDD patents from healthy controls in the training set (p<0.0001) as well as in the validation set (P=0.011; Table 3; FIG. 3). Analysis of the qPCR data for the training set and validation set of MDD patients and controls showed that this technically-independent and laboratory-convenient method could produce similar results to that with microarray analysis, as was evident from the significant correlation for the MDD-marker values obtained in qPCR and microarray analysis (Spearman's rho 0.489, P<0.001; FIG. 4A). In agreement with our microarray results, a significant separation between MDD patients and healthy controls was observed using qPCR for both the training and the validation set (P=0.007, P=0.019, respectively; FIG. 4B, Table 3). The correlation between Inventory of Depressive Symptomatology (IDS) and MDD-marker was significant (Spearman's rho 0.538, P<0.0001, 67 subjects). No significant correlation was found for any of the other parameters, e.g. smoking, age of onset of smoking, nicotine dependence, sex, age, and age of onset of MDD.

### Comment

At present, laboratory blood tests to support MDD diagnosis are not available. Linking gene expression profiling in whole blood after ex vivo LPS stimulation with clinical data rapidly identified diagnostic biomarkers of MDD. These were confirmed in an independent validation set, as well as by qPCR, the latter being an independent and convenient low-cost method.

To ensure studying disease state, rather than trait, we have included both single episode and recurrent cases for which the presence of a current major depressive episode was obligatory. Moreover, we have excluded controls with a history of MDD. In addition, we have studied the correlation between our MDD-marker and depression severity (IDS). Since co-morbid physical disorders and use of medication are common in most MDD cohorts, the MDD subjects in the present study may not entirely reflect MDD patients in day-to-day clinical practice. Therefore, the MDD-marker that we present in this study should be considered as a first step toward the use of such a biomarker in the general population.

An important feature of a biomarker is sensitivity and specificity of detection. The χ2-test is indicative for the high specificity and sensitivity of the marker (validation set qPCR p=0.012). The sensitivity (% MDD patients with positive marker outcome) of our MDD-marker is 76.9%, and the specificity (% controls with negative marker outcome) is 71.4% for the validation set using qPCR (FIG. 4B). These scores potentially contribute to a high predictive value. The biomarker described in this study might well be true endophenotypic marker. From independent work in twins and their siblings, we know that blood gene expression, and in particular genes that are regulated by ex vivo LPS application, have a strong genetic component (Spijker and Boomsma, unpublished research).

Apart from a possible genetic component that could explain the differences between MDD patients and healthy controls in relation to the MDD-marker presented, epigenetic differences could also play a role. In animal models^{30,31} and humans alike^{32,33}, variation in environment, e.g. early maternal care or prenatal SSRI exposure, may have a serious impact on the wellbeing of the offspring³⁴. Disrupted parenting is associated with differences in hypothalamic-pituitary-adrenal (HPA) stress response in the offspring, known to be mediated via changes in the epigenetic regulation of glucocorticoid receptor gene expression^{31,35}. In order to exclude a possibility of differential stress-response of leukocytes to the LPS-stimulus, we have analyzed our data for typical stress-response genes. In all individuals, stimulation of blood by LPS induced massive gene expression, among which are several cytokines (Table 6), such as TNF, NFkappaB, IL1a, IL6, 1110. However, none of these genes displayed a differential expression level between MDD patients and controls according to the limits set (−0.3>[MDD vs. control]>0.3; P<0.02). In addition, genes related to monoamine synthesis and release, such as serotonin and dopamine receptors, and the serotonin transporter, were not differentially expressed (Table 6) in either basal blood or LPS-stimulated blood between MDD patients and controls. Genes previously reported to be differentially expressed in whole blood, like 5HTT (SLC6A4), VEGF, PDE4B, HDAC, CREB and PDLIM5 (Table 6), were not confirmed in our NESDA cohort, possibly due to genetic differences with the Asian population in that study³⁶.

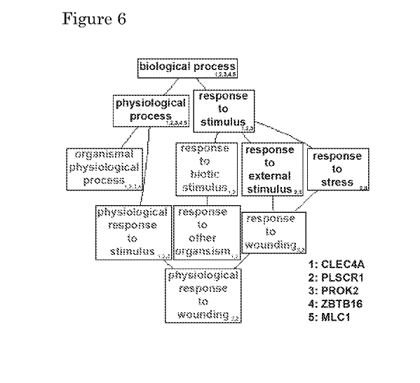

Although blood gene expression has the potential to reveal a biological pathway or mechanism that plays a role in neuropsychiatric disease⁸, the primary hallmark of a biomarker is the ability to classify subjects. In addition, performing gene expression analysis on LPS-stimulated blood may have further emphasized the finding of marker genes rather than biologically-relevant genes. An over-representation analysis of the marker genes compared with the genes present on the array revealed a significant result for several levels of the gene ontology class ‘biological process’ (FIG. 6). Further analysis showed that 6 out of 7 genes (Table 7) are related to the immune system and deal with cellular proliferation (CAPRIN1, PROK2 and ZBTB16) and differentiation (CLEC4A, KRT23, and PLSCR1).

Here, for the first time, we build a classifier gene set based on stimulated blood gene expression. The robustness of classification is exemplified in the low p-values obtained in the validation set (microarray), as well as the corroboration of the classifier using an independent technique (real-time qPCR). In general, markers for complex diseases are not simply present or absent. Rather, they have a wide range of values that overlap in persons with or without the disease, where the value typically increases progressively with increased risk or severity levels. The gene set, as specified for MDD in this study, has diagnostic value in determining disease state, because it correlated strongly with depression severity. The invention also applies for the predictive value of our marker for treatment outcome, as well as for the (re)occurrence of MDD in subjects that presently have no or only sub-threshold depressive disorders during the future 2-, or 4-year follow-up assessment of the NESDA study. The invention is also suited to test regular clinical screening tools. Obviously, a marker with predictive value has direct impact on day-to-day clinical practice, since it opens the door to prevention.

TABLE 1
Sample data as used in the total study, for the microarray and real-time
quantitative PCR (qPCR) using the training and validation set of MDD
patients (MDD) and healthy controls (Con). The number of participants
(N) in each group (total, females), as well as average age and IDS are
indicated. MDD patients were not significantly different (p = 0.345)
from healthy controls for nicotine dependence (Fagerstrom test). Only
one MDD patient (validation set) is a chronic cannabis user, and in total
5 persons (2 MDD, 3 control) reported the use of cannabis (7%) in the
last month before the interview and blood sampling. Further reports of
drug use during the last month were ecstasy (1 MDD patient), and
speed (1 control). None of the subjects reported use of cocaine, LSD
or heroin during the last month.

| | | | Array | | Array | | qPCR | | qPCR | |
|---|---|---|---|---|---|---|---|---|---|---|
| | Total | | training | | validation | | training | | validation | |
| | study | | set | | set | | set | | set | |
| Cases | MDD | Con | MDD | Con | MDD | Con | MDD | Con | MDD | Con |
| N total | 35 | 37 | 21 | 21 | 12 | 13 | 16 | 13 | 12 | 14 |
| Age (Y) | 42.7 | 42.4 | 42.3 | 41.9 | 44.9 | 41.5 | 41.1 | 44.8 | 44.9 | 45.6 |
| Female (n) | 23 | 23 | 14 | 14 | 8 | 9 | 11 | 8 | 7 | 8 |
| MDD, Single | 14 | 0 | 11 | 0 | 3 | 0 | 10 | 0 | 2 | 0 |
| Episode (n) | | | | | | | | | | |
| MDD, | 21 | 0 | 10 | 0 | 9 | 0 | 6 | 0 | 10 | 0 |
| Recurrent | | | | | | | | | | |
| (n) | | | | | | | | | | |
| IDS-score | 36 | 5 | 37 | 5 | 36 | 3 | 38 | 6 | 36 | 3 |
| (mean) | | | | | | | | | | |
| Comorbid | 20 | 0 | 12 | 0 | 5 | 0 | 12 | 0 | 5 | 0 |
| anxiety | | | | | | | | | | |
| disorder (n) | | | | | | | | | | |

TABLE 2
Candidate classifier genes from basal blood and LPS-stimulated
blood. Using the training set and the validation set of MDD patients and
controls, a set of 12 candidate classifier genes was measured for each
blood sample (basal and LPS-stimulated). Although these genes were
capable of discriminating cases of disease state in the training set, only
genes from the LPS-stimulated blood sample showed significant
differences in the validation set of MDD patients and controls. For all
data, the results (p-values) from the χ² test, Mann-Whitney
U-test, and Newman-Keuls test are indicated.

| Sample | χ² | MWU | t-test | MDD | Con |
|---|---|---|---|---|---|
| | Training set | | | | |
| Basal | 1.83E−04 | <0.0001 | 1.02E−05 | 21 | 21 |
| LPS | 5.57E−05 | <0.0001 | 1.31E−06 | | |
| | Validation set | | | | |
| Basal | 0.543 | 0.311 | 0.216 | 12 | 13 |
| LPS | 0.009 | 0.002 | 0.002 | | |

TABLE 3
Statistical analysis of MDD-marker score. Using both the training
and validation sets, the MDD-marker (composed of 7 genes that showed
similar results using microarray and real-time qPCR (FIG. 2)) was
measured. For all sets, p-values of the χ² test, Mann-Whitney U-test and
Newman-Keuls test indicated significance for the segregation of MDD
and controls.

| Sample | χ² | MWU | t-test | MDD | Con |
|---|---|---|---|---|---|
| | Training set | | | | |
| LPS | 0.000015 | <0.0001 | 0.00028 | 21 | 21 |
| array | | | | | |
| LPS | 0.00573 | 0.007 | 0.00398 | 13 | 16 |
| qPCR | | | | | |
| | Validation set | | | | |
| LPS | 0.022 | 0.011 | 0.016 | 12 | 13 |
| array | | | | | |
| LPS | 0.012 | 0.019 | 0.014 | 14 | 13 |
| qPCR | | | | | |

### References to the Example 2

1. Levinson D F. The genetics of depression: a review. Biol Psychiatry. 2006; 60(2):84-92.
2. Ebmeier K P, Donaghey C, Steele JD. Recent developments and current controversies in depression. Lancet. 2006; 367(9505):153-67.
3. Nestler E J, Barrot M, DiLeone R J, Eisch A J, Gold S J, Monteggia L M. Neurobiology of depression. Neuron. 2002; 34(1):13-25.
4. Kato T. Molecular genetics of bipolar disorder and depression. Psychiatry Clin Neurosci. 2007; 61(1):3-19.
5. Caspi A, Sugden K, Moffitt T E, Taylor A, Craig I W, Harrington H, McClay J, Mill J, Martin J, Braithwaite A, Poulton R. Influence of life stress on depression: moderation by a polymorphism in the 5-HTT gene. Science. 2003; 301(5631):386-9.
6. Gladkevich A, Kauffman H F, Korf J. Lymphocytes as a neural probe: potential for studying psychiatric disorders. Prog Neuropsychopharmacol Biol Psychiatry. 2004; 28(3):559-76.
7. Scherzer C R, Eklund A C, Morse L J, Liao Z, Locascio J J, Fefer D, Schwarzschild M A, Schlossmacher M G, Hauser M A, Vance J M, Sudarsky L R, Standaert D G, Growdon J H, Jensen R V, Gullans S R. Molecular markers of early Parkinson's disease based on gene expression in blood. Proc Natl Acad Sci USA. 2007; 104(3):955-60.
8. Le-Niculescu H, Kurian S M, Yehyawi N, Dike C, Patel S D, Edenberg H J, Tsuang M T, Salomon D R, Nurnberger J I, Jr., Niculescu A B. Identifying blood biomarkers for mood disorders using convergent functional genomics. Mol. Psychiatry. 2008.
9. Padmos R C, Hillegers M H, Knijff E M, Vonk R, Bouvy A, Staal F J, de Ridder D, Kupka R W, Nolen W A, Drexhage H A. A discriminating messenger RNA signature for bipolar disorder formed by an aberrant expression of inflammatory genes in monocytes. Arch Gen Psychiatry. 2008; 65(4):395-407.
10. Penninx B, Beekman A, Smit J, Zitman F, Nolen W, Spinhoven P, Cuijpers P, Jong Pd, Marwijk Hv, Assendelft W, Meer Kvd, Verhaak P, Wensing M, Graaf Rd, Hoogendijk W, Ormel J, Dyck Rv. The Netherlands Study of Depression and Anxiety (NESDA): Rationale, Objectives and Methods. Intern J Meth Psychiat Res. 2008; in press.
11. Sequeira A, Turecki G. Genome wide gene expression studies in mood disorders. Omics. 2006; 10(4):444-54.
12. Whitney A R, Diehn M, Popper S J, Alizadeh A A, Boldrick J C, Relman D A, Brown P O. Individuality and variation in gene expression patterns in human blood. Proc Natl Acad Sci USA. 2003; 100(4):1896-901.
13. Spijker S, van de Leemput J C, Hoekstra C, Boomsma D I, Smit A B. Profiling gene expression in whole blood samples following an in-vitro challenge. Twin Res. 2004; 7(6):564-70.
14. Guha M, Mackman N. LPS induction of gene expression in human monocytes. Cell Signal. 2001; 13(2):85-94.
15. Kalman J, Palotas A, Juhasz A, Rimanoczy A, Hugyecz M, Kovacs Z, Galsi G, Szabo Z, Pakaski M, Feher L Z, Janka Z, Puskas L G. Impact of venlafaxine on gene expression profile in lymphocytes of the elderly with major depression—evolution of antidepressants and the role of the “neuro-immune” system. Neurochem Res. 2005; 30(11):1429-38.
16. Irwin M. Immune correlates of depression. Adv Exp Med. Biol. 1999; 461:1-24.
17. Coplan J D, Tamir H, Calaprice D, DeJesus M, de la Nuez M, Pine D, Papp L A, Klein D F, Gorman J M. Plasma anti-serotonin and serotonin anti-idiotypic antibodies are elevated in panic disorder. Neuropsychopharmacology. 1999; 20(4):386-91.
18. Schwarz M J, Muller N, Riedel M, Ackenheil M. The Th2-hypothesis of schizophrenia: a strategy to identify a subgroup of schizophrenia caused by immune mechanisms. Med. Hypotheses. 2001; 56(4):483-6.
19. Stastny J, Konstantinidis A, Schwarz M J, Rosenthal N E, Vitouch O, Kasper S, Neumeister A. Effects of tryptophan depletion and catecholamine depletion on immune parameters in patients with seasonal affective disorder in remission with light therapy. Biol Psychiatry. 2003; 53(4):332-7.
20. Yirmiya R, Pollak Y, Morag M, Reichenberg A, Barak O, Avitsur R, Shavit Y, Ovadia H, Weidenfeld J, Morag A, Newman M E, Pollmacher T. Illness, cytokines, and depression. Ann N Y Acad. Sci. 2000; 917:478-87.
21. O'Connor J C, Lawson M A, Andre C, Moreau M, Lestage J, Castanon N, Kelley K W, Dantzer R. Lipopolysaccharide-induced depressive-like behavior is mediated by indoleamine 2,3-dioxygenase activation in mice. Mol. Psychiatry. 2008.
22. Boomsma D I, Willemsen G, Sullivan P F, Heutink P, Meijer P, Sondervan D, Kluft C, Smit G, Nolen W A, Zitman F G, Smit J H, Hoogendijk W J, van Dyck R, de Geus E J, Penninx B W. Genome-wide association of major depression: description of samples for the GAIN Major Depressive Disorder Study: NTR and NESDA biobank projects. Eur J Hum Genet. 2008; 16(3):335-42.
23. Robins L N, Wing J, Wittchen H U, Helzer J E, Babor T F, Burke J, Farmer A, Jablenski A, Pickens R, Regier D A, et al. The Composite International Diagnostic Interview. An epidemiologic Instrument suitable for use in conjunction with different diagnostic systems and in different cultures. Arch Gen Psychiatry. 1988; 45(12):1069-77.
24. American-Psychiatric-Association APA. Diagnostic and statistical manual of mental disorders, 4^{th} edition. Washington; 2002.
25. Kinouchi S, Iga J, Ueno S, Yamauchi K, Numata S, Song H, Sumitani S, Shibuya-Tayoshi S, Haku M, Yasui T, Irahara M, Morita K, Rokutan K, Ohmori T. FKBP5, SERT and COMT mRNA expressions in the peripheral leukocytes during menstruation cycle in healthy reproductive females. Neurosci Lett. 2008; 434(1):124-8.
26. Tibshirani R, Hastie T, Narasimhan B, Chu G. Diagnosis of multiple cancer types by shrunken centroids of gene expression. Proc Natl Acad Sci USA. 2002; 99(10):6567-72.
27. Iga J, Ueno S, Yamauchi K, Numata S, Tayoshi-Shibuya S, Kinouchi S, Nakataki M, Song H, Hokoishi K, Tanabe H, Sano A, Ohmori T. Gene expression and association analysis of vascular endothelial growth factor in major depressive disorder. Prog Neuropsychopharmacol Biol Psychiatry. 2007; 31(3):658-63.
28. Iga J, Ueno S, Yamauchi K, Numata S, Kinouchi S, Tayoshi-Shibuya S, Song H, Ohmori T. Altered HDAC5 and CREB mRNA expressions in the peripheral leukocytes of major depression. Prog Neuropsychopharmacol Biol Psychiatry. 2007; 31(3):628-32.
29. Lee S. Mistakes in validating the accuracy of a prediction classifier in high-dimensional butsmall-sample microarray data. Stat Methods Med. Res. 2008.
30. Newport D J, Stowe Z N, Nemeroff C B. Parental depression: animal models of an adverse life event. Am J Psychiatry. 2002; 159(8):1265-83
31. Francis D, Diorio J, Liu D, Meaney M J. Nongenomic transmission across generations of maternal behavior and stress responses in the rat. Science. 1999; 286(5442):1155-8.
32. Lovejoy M C, Graczyk P A, O'Hare E, Neuman G. Maternal depression and parenting behavior: a meta-analytic review. Clin Psychol Rev. 2000; 20(5):561-92.
33. Weissman M M, Jensen P. What research suggests for depressed women with children. J Clin Psychiatry. 2002; 63(7):641-7.
34. Oberlander T F, Grunau R, Mayes L, Riggs W, Rurak D, Papsdorf M, Misri S, Weinberg J. Hypothalamic-pituitary-adrenal (HPA) axis function in 3-month old infants with prenatal selective serotonin reuptake inhibitor (SSRI) antidepressant exposure. Early Hum Dev. 2008.
35. Oberlander T F, Weinberg J, Papsdorf M, Grunau R, Misri S, Devlin A M. Prenatal exposure to maternal depression, neonatal methylation of human glucocorticoid receptor gene (NR3C1) and infant cortisol stress responses. Epigenetics. 2008; 3(2):97-106.
36. Iga J, Ueno S, Ohmori T. Molecular assessment of depression from mRNAs in the peripheral leukocytes. Ann Med. 2008; 40(5):336-42.

### Supplementary Information

### Results

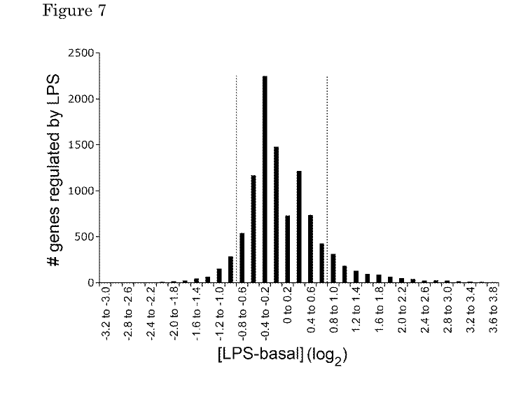

For the total set of arrays, filtering resulted in ˜24000 probes (˜60%) with a present-call. When the LPS sample was compared with the basal sample [LPS-basal] for all arrays, about 1700 probes (7%) showed significant regulation (FDR<0.0001; −0.8>[LPS-basal] (log 2)>0.8), with 65% of these up-regulated and 35% down-regulated (FIG. 7).

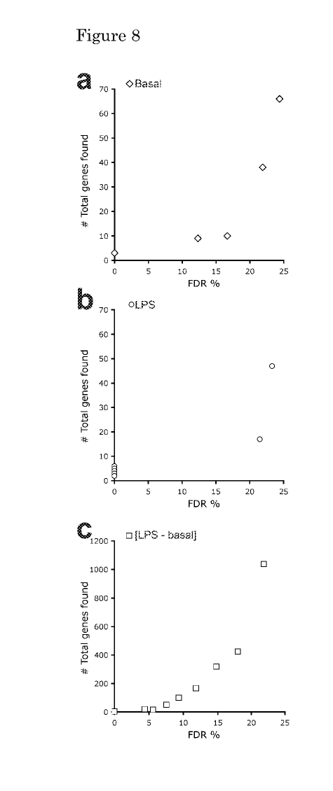

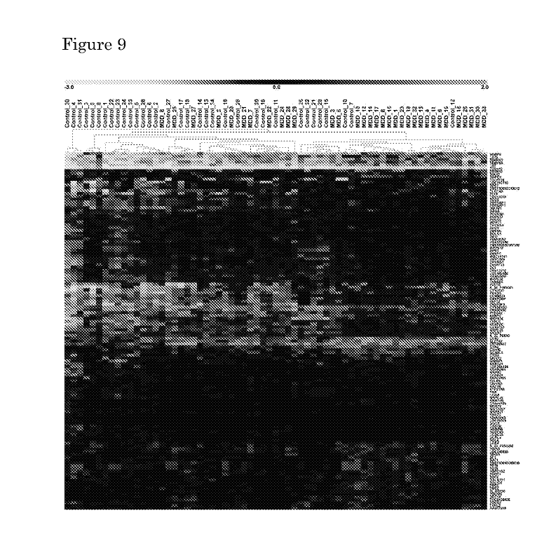

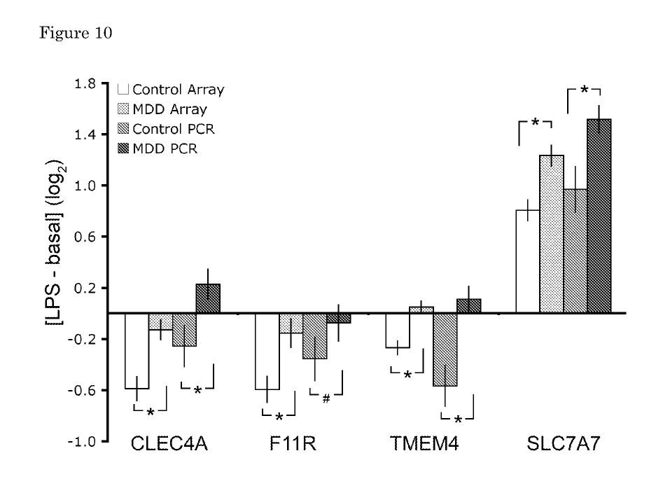

As a first step to analyze whether our data had the potential to discriminate for disease state and to be able to use a classifier approach (see below), we examined the number of genes that were significantly different in expression level between MDD patients and healthy controls (43 and 44 subjects, respectively). The differentially expressed genes that are discriminative for disease state were analyzed using a false-discovery rate (FDR) analysis. Notably, a low number (3 genes) of significant genes (FDR<0.1) were detected when comparing untreated blood samples (basal blood) (FIG. 8A) or blood samples that were stimulated with LPS (6 genes, FIG. 8B). However, when LPS-induced gene expression values—[LPS-basal] (ratio) for each patient—were compared, this resulted in 126 genes differentially expressed in MDD patients compared with healthy controls (FIG. 8C). Plots of the total number of genes found vs. the FDR (FIG. 8) show that stimulation of blood with a challenger (FIG. 8B,C) is able to reveal more differences between MDD patients and controls. In all samples, these genes were able to segregate blood samples based on disease state (62-90% correct, Table 8, FIG. 9) using several gene-grouping algorithms (hierarchical clustering, self-organizing maps (SOMs), and K-mean clustering). Real-time quantitative PCR (qPCR) (FIG. 10) for a set of 4 genes corroborated the differential expression between MDD and controls. Together, these results show that MDD patients and controls could be distinguished based on gene expression profiles in whole blood, indicating the possibility to find disease-markers. In addition, application of a robust challenge to blood samples may have an improved classification potential to discriminate subjects based on disease state.

In order to find the molecular marker for MDD, we used the Prediction Analysis of Microarrays (PAM)¹ analysis tool as described in the main text. Because genes with proven ability to classify samples do not necessarily need to be the most significant differentially expressed genes, we used all three data sets (gene expression from basal blood, LPS-stimulated blood, and the [LPS-basal] expression) in the classification algorithm. At the minimum misclassification error of 43.5%, 28.5%, 36%, the PAM analysis resulted in 160, 110, and 218 genes that classified subjects into two groups (MDD patients vs. control) for the three data sets used (basal, LPS and [LPS-basal], respectively). After reduction of this set of genes, 4 genes present in the LPS sample and one in the basal sample also scored relatively high as classifier in the [LPS-basal] sample. However, the final set of genes was selected based on participation from independent data sets.

TABLE 8
Clustering algorithms to segregate
MDD patients from healthy controls.

| Data set | Clustering | Hierachical | SOM | K-means |
|---|---|---|---|---|
| basal | Control | 65.1 | 70.3 | 67.6 |
| | MDD | 75.0 | 76.7 | 73.3 |
| LPS | Control | 75.8 | 73.1 | 92.9 |
| | MDD | 73.5 | 63.4 | 60.4 |
| [LPS-basal] | Control | 72.2 | 75.8 | 78.1 |
| | MDD | 73.3 | 73.5 | 74.3 |

Based on differentially expressed genes (FDR < 0.1%) between MDD and control, samples from 33 MDD patients and 34 healthy controls were clustered into two groups using data from basal blood (3 genes), LPS-stimulated blood (LPS; 6 genes), and from the comparison of the LPS with basal sample ([LPS-basal], 126 genes). The % of correct subjects assigned to each of the 2 nodes is displayed based on disease state. A graphical display of hierarchical clustering for [LPS-basal] is shown in FIG. 9.

### REFERENCES

1. Tibshirani R, Hastie T, Narasimhan B, Chu G. Diagnosis of multiple cancer types by shrunken centroids of gene expression. Proc Natl Acad Sci USA. 2002; 99(10):6567-72.

### Supplementary Information

TABLE 4
MDD patients and controls do not differ in leukocyte count. From
several samples of the validation set, leukocyte numbers (# × 10⁹/liter
blood) were analyzed. Different statistical tests (Mann-Whitney U test:
p-value and z-score, Newman-Keuls: t-test) show that there is no
difference in this parameter. There is no correlation (Spearman rank)
with the MDD marker value (P = 0.688).

| Sample | Average | Median | SEM | n | MWU-p | MWU-z | t-test |
|---|---|---|---|---|---|---|---|
| MDD | 6.20 | 5.4 | 1.02 | 6 | 0.905 | −0.258 | 0.962 |
| Control | 6.27 | 5.8 | 0.90 | 3 | | | |

TABLE 5
Individual MDD-patient characteristics. For all MDD patients used
in the study the age, gender, type of episode, the IDS, and co-morbid
anxiety disorders are indicated. Single episode: presence of a single
Major Depressive episode. Recurrent: presence of two or more Major
Depressive Episodes - with an interval of more than two months in
which criteria are not met for a Major Depressive episode). GAD:
Generalized Anxiety Disorder. Array training set: MDD 01-21, Array
validation set: MDD 22-33, qPCR training set: MDD 01-07, MDD12-17,
MDD 19-21, qPCR validation set MDD 22-33, MDD 35.

| | | | Single Episode | IDS- | Co-morbid Anxiety |
|---|---|---|---|---|---|
| Cases | Age | Gender | or Recurrent | score | Disorders |
| MDD01 | 46 | Male | Single Episode | 34 | GAD |
| MDD02 | 44 | Female | Single Episode | 45 | GAD |
| MDD03 | 29 | Female | Single Episode | 57 | GAD |
| MDD04 | 30 | Female | Single Episode | 41 | No |
| MDD05 | 38 | Female | Single Episode | 24 | GAD |
| MDD06 | 40 | Female | Recurrent | 48 | GAD |
| MDD07 | 47 | Female | Recurrent | 41 | GAD/Panic Disorder |
| MDD08 | 63 | Male | Recurrent | 33 | No |
| MDD09 | 43 | Male | Single Episode | 31 | GAD |
| MDD10 | 57 | Female | Recurrent | 22 | GAD/Panic Disorder |
| MDD11 | 38 | Female | Recurrent | 39 | Panic Disorder |
| MDD12 | 35 | Female | Recurrent | 26 | Social Phobia |
| MDD13 | 32 | Female | Single Episode | 41 | No |
| MDD14 | 45 | Male | Recurrent | 31 | Social Phobia |
| MDD15 | 60 | Male | Single Episode | 34 | No |
| MDD16 | 40 | Female | Single Episode | 41 | Panic Disorder |
| MDD17 | 22 | Female | Recurrent | 36 | GAD |
| MDD18 | 30 | Female | Recurrent | 33 | No |
| MDD19 | 55 | Male | Single Episode | 50 | GAD |
| MDD20 | 54 | Female | Single Episode | 32 | GAD/Social Phobia |
| MDD21 | 35 | Male | Recurrent | 31 | No |
| MDD22 | 63 | Male | Recurrent | 40 | No |
| MDD23 | 51 | Female | Recurrent | 47 | GAD/Panic Disorder |
| MDD24 | 60 | Male | Recurrent | 31 | No |
| MDD25 | 39 | Female | Recurrent | 40 | No |
| MDD26 | 39 | Female | Single Episode | 40 | Social Phobia |
| MDD27 | 41 | Male | Recurrent | 25 | No |
| MDD28 | 55 | Female | Recurrent | 28 | No |
| MDD29 | 29 | Male | Single Episode | 31 | No |
| MDD30 | 25 | Female | Recurrent | 27 | Social Phobia |
| MDD31 | 35 | Female | Recurrent | 44 | GAD/Social Phobia |
| MDD32 | 47 | Female | Recurrent | 55 | GAD |
| MDD33 | 55 | Male | Recurrent | 26 | No |
| MDD34 | 32 | Female | Single Episode | 28 | No |
| MDD35 | 34 | Female | Recurrent | 33 | No |

TABLE 6
Stress-response and monoamine genes are not differentially-
regulated between MDD patients and controls. Among the 1725 genes (see
FIG. 5) that were regulated significantly by LPS (FDR < 0.0001; −0.8 > r >
0.8; bold), were several stress response genes (C) known from literature, e.g.
cytokines. For this set of genes, the differential expression between MDD
patients and controls in basal and LPS-stimulated blood is presented
(averaged expression (log₂) for different probes present). However, none of
these genes showed a differential expression with respect to disease status (−0.3 >
[MDD vs. control] > 0.3; P < 0.02). As a positive control, the amount of
regulation due to stimulation by LPS (LPS vs. basal for all cases studied) is
given. This indicated that blood cells were indeed responsive to LPS. The
number of probes present for each gene is indicated. A search for “serotonin”
and “monoamine” resulted in monoamine-related genes (M) like serotonin
and dopamine receptors. Neither these, nor the epigenetically-related (Epi)
glucocorticoid receptor (NR3C1), nor genes found to be regulated in a
Japanese cohort (Jp) are differentially expressed between MDD patients and
controls in basal and LPS-stimulated blood. Genes with a modest LPS
induction (FDR < 0.0001; −0.5 > r > 0.5) are indicated (italics).

| | | | Basal | | LPS-stimulated | | |
|---|---|---|---|---|---|---|---|
| Gene | Gene | # | MDD vs | | MDD vs | | LPS vs basal |
| symbol | type | probes | Control | t-test | Control | t-test | All cases |
| CD69 | C | 10 | −0.09 | 0.239 | −0.27 | 0.389 | 1.15 |
| CD83 | C | 1 | −0.05 | 0.665 | −0.14 | 0.508 | 3.59 |
| CXCL2 | C | 2 | 0.00 | 0.945 | 0.00 | 0.903 | 3.91 |
| DUSP2 | C | 2 | −0.02 | 0.865 | −0.05 | 0.733 | 2.69 |
| IFITM1 | C | 1 | 0.03 | 0.834 | 0.11 | 0.407 | 1.14 |
| IFITM3 | C | 1 | 0.01 | 0.939 | 0.21 | 0.213 | 0.92 |
| IFNGR1 | C | 1 | −0.02 | 0.905 | 0.20 | 0.328 | −1.39 |
| IFNGR2 | C | 1 | 0.04 | 0.742 | 0.10 | 0.575 | 1.23 |
| IL10 | C | 1 | 0.06 | 0.234 | 0.28 | 0.056 | 1.52 |
| IL12B | C | 1 | −0.01 | 0.750 | −0.35 | 0.201 | 1.35 |
| IL17R | C | 1 | 0.08 | 0.540 | 0.06 | 0.662 | −1.14 |
| IL1A | C | 1 | 0.05 | 0.280 | −0.11 | 0.690 | 2.08 |
| IL1B | C | 1 | −0.06 | 0.725 | −0.36 | 0.212 | 5.77 |
| IL23A | C | 1 | −0.06 | 0.461 | −0.51 | 0.076 | 1.72 |
| IL6 | C | 1 | −0.02 | 0.882 | −0.29 | 0.519 | 5.66 |
| IL6R | C | 1 | −0.07 | 0.480 | 0.10 | 0.425 | −1.09 |
| IL8 | C | 1 | −0.07 | 0.337 | −0.18 | 0.637 | 1.48 |
| IL8RA | C | 1 | 0.10 | 0.469 | 0.07 | 0.460 | −2.54 |
| IL8RB | C | 2 | 0.05 | 0.675 | −0.04 | 0.608 | −2.23 |
| MX2 | C | 2 | 0.04 | 0.791 | 0.12 | 0.484 | 2.52 |
| MYD88 | C | 1 | 0.17 | 0.249 | 0.16 | 0.319 | 1.12 |
| NFKB1 | C | 1 | −0.07 | 0.403 | −0.16 | 0.215 | 3.13 |
| NFKB2 | C | 1 | 0.03 | 0.745 | −0.01 | 0.941 | 2.16 |
| NFKBIA | C | 1 | 0.07 | 0.607 | 0.12 | 0.487 | 3.49 |
| PTEN | C | 2 | 0.10 | 0.466 | 0.04 | 0.699 | −1.04 |
| TGFBI | C | 1 | −0.23 | 0.049 | −0.28 | 0.063 | −2.72 |
| TNF | C | 2 | 0.02 | 0.782 | −0.24 | 0.353 | 3.09 |
| TNFAIP3 | C | 2 | −0.06 | 0.498 | −0.13 | 0.356 | 3.07 |
| TNFAIP6 | C | 1 | 0.04 | 0.784 | 0.02 | 0.908 | 4.90 |
| AANAT | M | 1 | −0.09 | 0.308 | −0.07 | 0.383 | 0.10 |
| ADCYAP1R1 | M | 1 | −0.03 | 0.546 | −0.06 | 0.152 | 0.01 |
| ALG9 | M | 2 | −0.01 | 0.689 | 0.01 | 0.821 | 0.09 |
| AOC3 | M | 1 | 0.07 | 0.333 | 0.01 | 0.689 | −0.50 |
| AOF2 | M | 1 | −0.01 | 0.717 | 0.02 | 0.601 | −0.06 |
| ATP7A | M | 1 | 0.00 | 0.880 | −0.01 | 0.763 | −0.11 |
| CANX | M | 2 | −0.02 | 0.619 | 0.09 | 0.417 | 0.01 |
| CAV1 | M | 1 | 0.10 | 0.596 | −0.01 | 0.974 | 0.20 |
| CC2D1A | M | 1 | −0.04 | 0.412 | −0.02 | 0.745 | −0.27 |
| CDCA7L | M | 1 | −0.07 | 0.342 | −0.16 | 0.038 | −0.16 |
| CHRNA4 | M | 2 | 0.01 | 0.763 | −0.04 | 0.735 | 0.16 |
| COMT | M | 1 | −0.13 | 0.189 | −0.07 | 0.356 | −0.58 |
| CYP2D6 | M | 2 | −0.05 | 0.423 | −0.08 | 0.219 | −0.05 |
| DRD2 | M | 1 | 0.00 | 0.962 | −0.06 | 0.486 | 0.12 |
| DRD4 | M | 2 | 0.01 | 0.631 | −0.02 | 0.864 | −0.20 |
| EDN1 | M | 1 | 0.01 | 0.869 | −0.20 | 0.168 | 2.59 |
| EGR1 | M | 1 | −0.08 | 0.727 | −0.03 | 0.877 | 0.65 |
| FCER1A | M | 1 | −0.19 | 0.159 | −0.19 | 0.005 | −1.53 |
| FEV | M | 1 | 0.01 | 0.630 | 0.00 | 0.992 | 0.06 |
| FOS | M | 1 | −0.07 | 0.780 | −0.06 | 0.736 | −1.64 |
| GNA11 | M | 2 | 0.03 | 0.424 | 0.04 | 0.423 | 0.04 |
| GNAO1 | M | 1 | 0.02 | 0.589 | 0.00 | 0.919 | 0.05 |
| GNAQ | M | 1 | 0.01 | 0.530 | −0.01 | 0.518 | −0.10 |
| GNB3 | M | 1 | 0.01 | 0.855 | 0.00 | 0.974 | 0.15 |
| HSPA5 | M | 2 | −0.07 | 0.462 | −0.15 | 0.243 | 0.42 |
| HTR1B | M | 1 | 0.00 | 0.995 | −0.13 | 0.253 | 0.10 |
| HTR1D | M | 1 | 0.00 | 0.910 | −0.01 | 0.762 | 0.02 |
| HTR3A | M | 1 | −0.04 | 0.543 | −0.05 | 0.520 | 0.09 |
| HTR3E | M | 1 | −0.01 | 0.452 | −0.02 | 0.288 | 0.04 |
| HTR7 | M | 1 | −0.04 | 0.069 | 0.00 | 0.935 | −0.06 |
| HTRA3 | M | 1 | 0.07 | 0.050 | 0.11 | 0.092 | 0.12 |
| IL4I1 | M | 1 | −0.10 | 0.499 | −0.15 | 0.593 | 4.04 |
| INPP1 | M | 1 | 0.03 | 0.424 | 0.04 | 0.510 | 0.27 |
| ITGA2B | M | 2 | −0.10 | 0.455 | −0.20 | 0.122 | −0.20 |
| ITGB3 | M | 2 | −0.04 | 0.635 | −0.15 | 0.070 | −0.23 |
| JAK2 | M | 1 | 0.18 | 0.218 | 0.14 | 0.239 | −0.38 |
| JUN | M | 1 | −0.05 | 0.701 | −0.32 | 0.071 | 2.29 |
| KLF11 | M | 2 | −0.05 | 0.211 | −0.06 | 0.176 | −0.12 |
| MAOA | M | 1 | −0.01 | 0.759 | −0.02 | 0.719 | 0.16 |
| MAPK1 | M | 2 | 0.12 | 0.137 | 0.08 | 0.256 | −0.64 |
| MAPK14 | M | 3 | 0.03 | 0.644 | 0.05 | 0.618 | −0.57 |
| MAPK3 | M | 1 | 0.11 | 0.132 | 0.03 | 0.662 | −0.41 |
| PAOX | M | 1 | −0.10 | 0.082 | 0.05 | 0.490 | −0.12 |
| PARK2 | M | 1 | −0.01 | 0.884 | −0.04 | 0.283 | 0.08 |
| PRKCD | M | 1 | −0.03 | 0.770 | 0.01 | 0.963 | 0.40 |
| ROCK1 | M | 1 | 0.02 | 0.564 | −0.01 | 0.740 | −0.13 |
| SIGLEC7 | M | 2 | −0.02 | 0.815 | 0.06 | 0.450 | −0.51 |
| SIGLEC9 | M | 1 | 0.06 | 0.326 | 0.07 | 0.611 | 0.41 |
| SLC29A4 | M | 1 | 0.04 | 0.063 | 0.01 | 0.633 | 0.07 |
| SLC6A4 | M, Jp | 1 | −0.02 | 0.315 | −0.04 | 0.033 | −0.01 |
| SMOX | M | 1 | −0.03 | 0.844 | −0.15 | 0.245 | 0.01 |
| SNX27 | M | 2 | −0.02 | 0.810 | 0.07 | 0.305 | −0.92 |
| SP1 | M | 2 | 0.01 | 0.727 | 0.01 | 0.721 | 0.01 |
| SP3 | M | 2 | 0.09 | 0.537 | 0.01 | 0.516 | −0.37 |
| SRC | M | 1 | 0.06 | 0.518 | 0.06 | 0.525 | 1.08 |
| STX1A | M | 2 | −0.03 | 0.589 | −0.01 | 0.808 | 0.41 |
| SYK | M | 1 | −0.06 | 0.494 | 0.07 | 0.514 | −0.94 |
| YWHAQ | M | 4 | 0.16 | 0.262 | 0.26 | 0.166 | −0.25 |
| NR3C1 | Epi | 2 | 0.01 | 0.440 | 0.22 | 0.210 | 0.25 |
| PDLIM5 | Jp | 4 | 0.00 | 0.558 | 0.10 | 0.438 | 0.16 |
| CREB1 | Jp | 2 | 0.07 | 0.230 | 0.16 | 0.047 | 0.02 |
| PDE4B | Jp | 2 | 0.05 | 0.572 | 0.18 | 0.514 | 1.21 |
| HDAC5 | Jp | 2 | −0.17 | 0.155 | −0.06 | 0.525 | −0.94 |
| VEGF | Jp | 4 | 0.03 | 0.615 | 0.01 | 0.615 | 0.60 |

TABLE 7
Initial set of classifier genes for MDD. For the top-12 genes from the PAM analysis of basal and LPS-stimulated blood, the gene symbol, and gene ontology (GO) annotation (level 3) for biological process (upper)
and molecular function (lower) is given. Genes taken for the final MDD marker set as analyzed in LPS-stimulated blood is indicated in bold. Note that for some genes no GO annotation is known.

| | | | | | | | | | | | | | | Channel or | |
|---|---|---|---|---|---|---|---|---|---|---|---|---|---|---|---|
| | | | | | Nucleic | | | | | Structural | Transcription | Transcriptional | Carbohydrate | pore class | Ion |
| | | Antigen | Peroxidase | Carbohydrate | acid | Ion | Protein | Oxireductase | Receptor | constituent of | factor | repressor | transporter | transporter | transporter |
| | | binding | activity | binding | binding | binding | binding | activity | activity | ribosome | activity | activity | activity | activity | activity |
| Basal top 12 | A_24_P789842 | | | | | | | | | | | | | | |
| | CORO1A | | | | | | + | | | | | | | | |
| | FCN2 | + | | + | | + | | | | | | | | | |
| | KRT23 | | | | | | | | | | | | | | |
| | MLC1 | | | | | | | | | + | | | | + | + |
| | NRGN | | | | | | + | | | | | | | | |
| | PLXNB2 | | | | | | | | + | | | | | | |
| | PPBP | | | | | | + | | | | | | + | | |
| | PTGS1 | | + | | | + | | + | | | | | | | |
| | RNPC1 | | | | | | | | | | | | | | |
| | SOX4 | | | | + | | | | | | + | | | | |
| | VAMP8 | | | | | | | | | | | | | | |
| | AL833005 | | | | | | | | | | | | | | |
| | CAPRIN1 | | | | | | | | | | | | | | |
| | CLEC4A | | | + | | | | | + | | | | | | |
| | FLJ23556 | | | | | | | | | | | | | | |
| | ITGB3 | | | | | | + | | + | | | | | | |
| | KRT23 | | | | | | | | | | | | | | |
| | LMNA | | | | | | + | | | | | | | | |
| | MLC1 | | | | | | | | | + | | | | + | + |
| | NBR1 | | | | | + | | | | | | | | | |
| | PLSCR1 | | | | | + | + | | | | | | | | |
| | PROK2 | | | | | | + | | | | | | | | |
| | ZBTB16 | | | | + | + | + | | | | | + | | | |

| | | Lipid | | | | Cellular | Regulation | Anatomical | Regulation | | | | | |
|---|---|---|---|---|---|---|---|---|---|---|---|---|---|---|
| | | transporter | | Cell | Cell | physiological | of cellular | structure | of | | | | | |
| | | activity | Cell adhesion | communication | differentiation | process | process | development | development | Coagulation | Death | Homeostasis | Localization | Locomotion |
| Basal top 12 | A_24_P789842 | | | | | | | | | | | | | |
| | CORO1A | | | | + | | | | | | | + | + | |
| | FCN2 | | | | + | | | | | | | + | | |
| | KRT23 | | | | | | | | | | | | | |
| | MLC1 | | | | + | | | | | | | + | | |
| | NRGN | | + | | | | + | | | | | | | |
| | PLXNB2 | | | | | | | | | | | | | |
| | PPBP | | | | + | + | | | | | | + | | |
| | PTGS1 | | | + | + | | | | | | | | | |
| | RNPC1 | | | | | | | | | | | | | |
| | SOX4 | | | | + | + | | | | | | | | |
| | VAMP8 | | | | + | | | | | | | + | | |
| | AL833005 | | | | | | | | | | | | | |
| | CAPRIN1 | | | | | | | | | | | | | |
| | CLEC4A | + | + | | | | | | | | | | | |
| | FLJ23556 | | | | | | | | | | | | | |
| | ITGB3 | + | + | | | | | | + | | | | | |
| | KRT23 | | | | | | | | | | | | | |
| | LMNA | | | | | | | | | | | | | |
| | MLC1 | | | | + | | | | | | | + | | |
| | NBR1 | | | | | | | | | | | | | |
| | PLSCR1 | + | | | | + | | | | + | + | | | |
| | PROK2 | | + | | + | + | + | | | + | + | | | |
| | ZBTB16 | | | + | + | + | + | + | | | | | | |

| | | | | | Regulation | | Negative | Positive | | | | | | | | |
|---|---|---|---|---|---|---|---|---|---|---|---|---|---|---|---|---|
| | | | Organismal | Physiological | of | | regulation | regulation | Regulation | | | | Response | Response | Response | |
| | | | physiological | response | physiological | Rhythmic | biological | biological | catalytic | Sexual | | | ro biotic | ro chemical | ro external | Response |
| | | Metabolism | process | to stimulus | process | process | process | process | activity | reproduction | Behavior | | stimuli | stimuli | stimuli | to stress |
| Basal | A_24_P789842 | | | | | | | | | | | | | | | |
| top 12 | CORO1A | | | | | | | | | | | | | | | |
| | FCN2 | | + | + | | | | | | | | | + | | | |
| | KRT23 | | | | | | | | | | | | | | | |
| | MLC1 | + | | | | | | | | | | | | | | |
| | NRGN | | | | | | | | | | | | | | | |
| | PLXNB2 | | | | | | | | | | | | | | | |
| | PPBP | | + | + | + | | | | | | + | + | + | + | + | |
| | PTGS1 | + | + | | | | | | | | | | | | | |
| | RNPC1 | | | | | | | | | | | | | | | |
| | SOX4 | + | | | + | | | | | | | | | | | |
| | VAMP8 | + | | | | | | | | | | | | | | |
| | AL833005 | | | | | | | | | | | | | | | |
| | CAPRIN1 | | | | | | | | | | | | | | | |
| | CLEC4A | | + | + | | | | | | | | | + | | | |
| | FLJ23556 | | | | | | | | | | | | | | | |
| | ITGB3 | | + | + | | | | | | | | | | | + | + |
| | KRT23 | | | | | | | | | | | | | | | |
| | LMNA | | | | | | | | | | | | | | | |
| | MLC1 | | | | | | | | | | | | | | | |
| | NBR1 | | | | | | | | | | | | | | | |
| | PLSCR1 | | + | + | | | | | | | | | + | | + | + |
| | PROK2 | | + | + | + | + | + | + | + | + | + | | | + | + | + |
| | ZBTB16 | + | + | | + | | + | | | | | | | | | |

TABLE 9

| | Alternative | Genesymbol or | | | | |
|---|---|---|---|---|---|---|
| Gene No | Gene No | Gene ID | Alternative ID | Unigene cluster | ProbeID | Gene Bank ID |
| 1 | 8 | HBG1 | | Hs.712539 | A_23_P53137, | NM_000559 |
| | | | | | A_23_P64539, | |
| | | | | | A_24_P289709 | |
| 2 | 4 | KRT23 | | Hs.9029 | A_23_P78248 | NM_015515 |
| 3 | 9 | AL833005 | DKFZp666D074 | Hs.675929 | A_32_P164917, | AL833005 |
| | | | | | A_32_P164916 | |
| 4 | 1 | CAPRIN1 | M11S1 | Hs.471818 | A_32_P235872, | NM_005898, NM_203364 |
| | | | | | A_23_P98722 | |
| 5 | 23 | ARAP3 | CENTD3 | Hs.25277 | A_23_P167389 | NM_022481 |
| 6 | 3 | PROK2 | | Hs.528665 | A_24_P97342 | NM_001126128, NM_021935 |
| 7 | 2 | ZBTB16 | | Hs.591945, | A_23_P104804 | NM_006006, NM_001018011 |
| | | | | Hs.682144 | | |
| 8 | 29 | F11R | | Hs.517293 | A_24_P319364, | NM_144503, NM_016946 |
| | | | | | A_24_P319369 | |
| 9 | 30 | FANCE | | Hs.302003 | A_23_P42335 | NM_021922 |
| 10 | 31 | LOC150166 | | Hs.48353 | A_32_P103815 | 150166 |
| 11 | 32 | CNPY2 | TMEM4 | Hs.8752 | A_23_P53288 | NM_014255 |
| 12 | 33 | SLC7A7 | | Hs.513147 | A_23_P99642 | NM_003982 |
| 13 | 5 | CLEC4A | | Hs.504657 | A_23_P48029 | NM_016184, NM_194447, |
| | | | | | | NM_194448, NM_194450 |
| 14 | 34 | NLRP3 | CIAS1 | Hs.159483 | A_23_P9883 | NM_001079821, |
| | | | | | | NM_004895, NM_183395 |
| 15 | 10 | RBM38 | RNPC1 | Hs.236361 | A_24_P14485, | NM_017495, NM_183425 |
| | | | | | A_23_P17430 | |
| 16 | 18 | CORO1A | | Hs.415067 | A_23_P106761 | NM_007074 |
| 17 | 35 | AMPD3 | | Hs.501890 | A_23_P116286, | NM_000480, |
| | | | | | A_24_P304154 | NM_001025389, |
| | | | | | | NM_001025390 |
| 18 | 36 | ARHGEF10L | | Hs.443460 | A_23_P386, | NM_018125, NM_001011722 |
| | | | | | A_23_P382 | |
| 19 | 37 | PRPSAP1 | | Hs.77498 | A_23_P15305 | NM_002766 |
| 20 | 38 | COMMD8 | | Hs.23956 | A_23_P44257 | NM_017845 |
| 21 | 39 | LMNA | | Hs.594444 | A_23_P34835, | NM_170707, NM_170708, |
| | | | | | A_24_P162718 | NM_005572 |
| 22 | 7 | MLC1 | | Hs.517729 | A_23_P211680 | NM_015166, NM_139202 |
| 23 | 66 | AK126405 | | Hs.197143 | A_24_P766716 | AK126405 |
| 24 | 67 | BC035647 | | Hs.656882 | A_23_P373126 | XM_001146616, |
| | | | | | | XM_001146687 |
| 25 | 68 | BI836739 | | Hs.173034 | A_32_P194704 | BI836739 |
| 26 | 69 | BRI3 | | Hs.567438 | A_23_P122915 | NM_015379 |
| 27 | 70 | C10orf125 | | Hs.155823 | A_23_P97952 | NM_198472, NM_001098483 |
| 28 | 71 | TMEM204 | C16orf30 | Hs.459652 | A_23_P37685 | NM_024600 |
| 29 | 72 | C20orf103 | | Hs.22920 | A_23_P40295 | NM_012261 |
| 30 | 73 | C22orf9 | | Hs.592207 | A_32_P88479 | NM_001009880, NM_015264 |
| 31 | 74 | NLRC4 | CARD12 | Hs.574741 | A_23_P119835 | NM_021209 |
| 32 | 75 | CD163 | | Hs.504641 | A_23_P33723 | NM_004244, NM_203416 |
| 33 | 76 | CDK4 | | Hs.95577 | A_23_P24997 | NM_000075 |
| 34 | 77 | CEACAM4 | | Hs.12 | A_24_P70480 | NM_001817 |
| 35 | 78 | CEBPB | | Hs.711943, | A_23_P411296 | NM_005194 |
| | | | | Hs.517106, | | |
| | | | | Hs.701858 | | |
| 36 | 79 | CHI3L1 | | Hs.382202 | A_23_P137665 | NM_001276 |
| 37 | 80 | CMTM3 | CKLFSF3 | Hs.298198 | A_23_P88865 | NM_001048251, NM_144601, |
| | | | | | | NM_181553, NM_181554 |
| 38 | 81 | CLDN5 | | Hs.505337 | A_23_P6321 | NM_003277 |
| 39 | 82 | KDELR2 | CR616528 | Hs.654552 | A_24_P410686 | NM_001100603, NM_006854 |
| 40 | 83 | C7orf42 | CR617678 | Hs.488478 | A_24_P346368 | NM_017994 |
| 41 | 84 | C9orf69 | CR602592 | Hs.287411 | A_23_P398372 | NM_152833 |
| 42 | 85 | CXCL5 | | Hs.89714 | A_24_P277367 | NM_002994 |
| 43 | 86 | DHRS3 | | Hs.289347 | A_23_P33759 | NM_004753 |
| 44 | 87 | DOK1 | | Hs.103854 | A_23_P5601 | NM_001381 |
| 45 | 88 | DOT1L | | Hs.713641 | A_23_P408768 | NM_032482 |
| 46 | 89 | EFNB1 | | Hs.144700 | A_24_P365807 | NM_004429 |
| 47 | 90 | EHD1 | | Hs.523774 | A_23_P52647 | NM_004429 |
| 48 | 91 | FLJ31306 | ENST00000316535 | Hs.531089 | A_24_P495122 | XM_001717968 |
| 49 | 92 | FAM108C1 | ENST00000258884, | Hs.459072 | A_32_P39093 | NM_021214 |
| | | | FLJ34461, | | | |
| | | | MGC131546 | | | |
| 50 | 93 | HSPC159 | ENST00000238875 | Hs.372208 | A_23_P210330 | NM_014181 |
| 51 | 94 | FAM13A1 | | Hs.97270 | A_23_P370651 | NM_014883, NM_001015045 |
| 52 | 95 | FAM78A | | Hs.143878, | A_23_P314250 | NM_033387 |
| | | | | Hs.704076 | | |
| 53 | 96 | FCN1 | | Hs.440898 | A_23_P157875, | NM_002003 |
| | | | | | A_23_P157879, | |
| | | | | | A_24_P263793 | |
| 54 | 24 | FCN2 | | Hs.54517 | A_23_P501732 | NM_004108, NM_015837 |
| 55 | 97 | C17orf59 | FLJ20014 | Hs.129563 | A_23_P152955 | NM_017622 |
| 56 | 98 | DDX60 | FLJ20035 | Hs.591710 | A_24_P334361, | NM_017631 |
| | | | | | A_23_P41470 | |
| 57 | 99 | PID1 | FLJ20701 | Hs.715695 | A_23_P21485 | NM_001100818, NM_017933 |
| 58 | 100 | hCG_1776259, | FLJ23556 | Hs.655463 | A_23_P149798 | XR_040924 |
| 59 | 101 | GIT2 | | Hs.434996 | A_23_P335848 | NM_014776, NM_057169, |
| | | | | | | NM_057170, NM_139201 |
| 60 | 102 | GLG1 | | Hs.201712 | A_23_P206510 | NM_012201 |
| 61 | 103 | GMPR2 | | Hs.368855 | A_24_P56467 | NM_001002000, |
| | | | | | | NM_001002001, |
| | | | | | | NM_001002002, NM_016576 |
| 62 | 104 | GRASP | | Hs.407202 | A_23_P105442 | NM_181711 |
| 63 | 105 | HLA-DMB | | Hs.654428 | A_32_P351968 | NM_002118 |
| 64 | 106 | ICAM2 | | Hs.431460 | A_23_P152655 | NM_001099786, |
| | | | | | | NM_001099787, |
| | | | | | | NM_001099788, |
| | | | | | | NM_001099789, NM_000873 |
| 65 | 107 | ID2 | | Hs.180919 | A_23_P143143 | NM_002166 |
| 66 | 108 | KCNE3 | | Hs.523899 | A_23_P24948 | NM_005472 |
| 67 | 109 | RIMBP3 | KIAA1666 | Hs.115429 | A_23_P154962 | NM_015672 |
| 68 | 110 | KIAA1949 | | Hs.696054 | A_23_P331479 | NM_133471 |
| 69 | 111 | KIAA2013 | | Hs.520094 | A_23_P51346 | NM_138346 |
| 70 | 112 | LAT2 | | Hs.647049 | A_23_P259621 | NM_032463, NM_032464, |
| | | | | | | NM_014146 |
| 71 | 113 | LFNG | | Hs.159142 | A_23_P8452 | NM_001040167, |
| | | | | | | NM_001040168 |
| 72 | 114 | LIMS3 | | Hs.535619 | A_23_P365685 | NM_033514 |
| 73 | 115 | LOC388524 | | Hs.560655 | A_23_P254415, | NM_001005472 |
| | | | | | A_24_P508410, | |
| | | | | | A_32_P219657 | |
| 76 | 116 | MAFB | | Hs.712609 | A_23_P17345 | NM_005461 |
| 77 | 117 | TMEM101 | MGC4251 | Hs.514211 | A_23_P15516 | NM_032376 |
| 78 | 118 | MRPS34 | | Hs.157160 | A_23_P163496 | NM_023936 |
| 79 | 40 | NDRG2 | | Hs.525205 | A_23_P37205 | NM_201536, NM_201537, |
| | | | | | | NM_201535, NM_201538, |
| | | | | | | NM_201539, NM_201540, |
| | | | | | | NM_201541, NM_016250 |
| 80 | 119 | NEURL | | Hs.594708 | A_23_P322562, | NM_004210 |
| | | | | | A_23_P138492 | |
| 81 | 120 | PF4 | | Hs.81564 | A_24_P79403 | NM_002619 |
| 82 | 6 | PLSCR1 | | Hs.130759 | A_23_P69109 | NM_021105 |
| 83 | 12 | PLXNB2 | | Hs.3989 | A_24_P70888, | NM_012401 |
| | | | | | A_32_P48397 | |
| 84 | 121 | PPIF | | Hs.381072 | A_23_P202104 | NM_005729 |
| 85 | 122 | PTP4A2 | | Hs.470477 | A_23_P23114 | NM_080391, NM_080392 |
| 86 | 123 | PYDC1 | | Hs.58314 | A_23_P407614 | NM_152901 |
| 87 | 124 | PYGO2 | | Hs.533597 | A_23_P411953 | NM_138300 |
| 88 | 125 | RAB37 | | Hs.592097 | A_23_P414654 | NM_175738, NM_001006638 |
| 89 | 126 | TRIM27 | RFP | Hs.440382 | A_32_P137035 | NM_006510 |
| 90 | 127 | RPL26 | | Hs.482144 | A_23_P33045, | NM_000987 |
| | | | | | A_32_P93782 | |
| 91 | 128 | RPL3 | | Hs.119598 | A_32_P209350, | NM_000967, NM_001033853 |
| | | | | | A_23_P68942 | |
| 92 | 41 | RPL34 | | Hs.438227 | A_24_P203909, | NM_000995, NM_033625 |
| | | | | | A_24_P303118, | |
| | | | | | A_23_P7221 | |
| 93 | 129 | RPL39 | | Hs.300141 | A_23_P34018, | NM_001000 |
| | | | | | A_32_P220307 | |
| 94 | 130 | RPS17 | | Hs.433427 | A_24_P418418, | NM_001021 |
| | | | | | A_23_P117721 | |
| 95 | 131 | RXRA | | Hs.590886 | A_23_P423197 | NM_002957 |
| 96 | 132 | BATF3 | SNFT | Hs.62919 | A_23_P160720 | NM_018664 |
| 97 | 13 | SOX4 | | Hs.699195 | A_23_P82169 | NM_003107 |
| 98 | 133 | SPG21 | | Hs.242458 | A_23_P147450 | NM_016630 |
| 99 | 22 | SPON2 | | Hs.302963 | A_23_P121533 | NM_001128325, NM_012445 |
| 100 | 134 | STAT4 | | Hs.80642 | A_23_P305198 | NM_003151 |
| 101 | 135 | TAF12 | | Hs.530251 | A_23_P63178 | NM_005644 |
| 102 | 136 | TNFAIP8L2 | | Hs.709522 | A_23_P46356 | NM_024575 |
| 103 | 137 | TRAF3IP3 | | Hs.147434 | A_23_P334414, | NM_025228, |
| | | | | | A_23_P323761 | |
| 104 | 138 | TSEN34 | | Hs.15580 | A_23_P130626 | NM_024075, NM_001077446 |
| 105 | 16 | VAMP8 | | Hs.714302 | A_23_P28434 | NM_003761 |
| 106 | 139 | ZDHHC7 | | Hs.592065 | A_23_P129389, | NM_017740 |
| | | | | | A_24_P373297 | |
| 107 | 11 | TLR1 | | Hs.654532 | A_23_P10873 | NM_003263 |
| 108 | 14 | TREML1 | | Hs.117331 | A_23_P156550 | NM_178174 |
| 109 | 15 | PTGS1 | | Hs.201978 | A_23_P216966 | NM_000962, NM_080591 |
| 110 | 17 | NRGN | | Hs.524116 | A_23_P116264 | NM_001126181, NM_006176 |
| 111 | 19 | NBR1 | | Hs.373818, | A_23_P207399 | NM_005899, NM_031858, |
| | | | | Hs.546264 | | NM_031862 |
| 112 | 20 | ITGB3 | | Hs.218040 | A_24_P318656 | NM_000212 |
| 113 | 21 | TCFL5 | | Hs.708155 | A_23_P143147 | NM_006602 |
| 114 | 25 | HLA-DRA | | Hs.520048 | A_32_P87697 | NM_019111 |
| 115 | 26 | SEPT5 | GP1BB | Hs.283743 | A_23_P29124 | NM_002688 |
| 116 | 27 | GAS2L1 | | Hs.322852 | A_23_P502710 | NM_006478, NM_152236, |
| | | | | | | NM_152237 |
| 117 | 28 | CLU | | Hs.436657 | A_23_P215913 | NM_001831, NM_203339 |
| 118 | 42 | RPL7 | | Hs.571841 | A_32_P155364 | NM_000971 |
| 119 | 43 | GNAZ | | Hs.584760 | A_23_P416581 | NM_002073 |
| 120 | 44 | SPOCD1 | | Hs.62604 | A_23_P431388 | NM_144569 |
| 121 | 45 | MYLK | | Hs.477375 | A_23_P143817 | NM_053025, NM_053026, |
| | | | | | | NM_053027, NM_053028, |
| | | | | | | NM_053031, NM_053032 |
| 122 | 46 | TUBA8 | | Hs.137400 | A_24_P160104 | NM_018943 |
| 123 | 47 | ITGA2B | | Hs.411312 | A_23_P77971 | NM_000419 |
| 124 | 48 | VCL | | Hs.643896 | A_24_P47182 | NM_014000, NM_003373 |
| 125 | 49 | ITGB5 | | Hs.536663 | A_23_P166633 | NM_002213 |
| 126 | 50 | TUBB1 | | Hs.592143 | A_23_P6034 | NM_030773 |
| 127 | 51 | VWF | | Hs.440848 | A_23_P105562 | NM_000552 |
| 128 | 52 | CMTM5 | CKLFSF5 | Hs.99272 | A_23_P106042 | NM_138460, NM_001037288 |
| 129 | 53 | S1PR1 | EDG1 | Hs.154210, | A_23_P404481 | NM_001400 |
| | | | | Hs.715612 | | |
| 130 | 54 | HEXIM2 | | Hs.56382 | A_23_P377214 | NM_144608 |
| 131 | 55 | ABCC3 | | Hs.463421 | A_23_P207507 | NM_003786 |
| 132 | 56 | CTBS | | Hs.706743 | A_23_P201368 | NM_004388 |
| 133 | 57 | AQP10 | | Hs.259048 | A_23_P126613 | NM_080429 |
| 134 | 58 | COX4I1 | | Hs.433419 | A_23_P141029 | NM_001861 |
| 135 | 59 | HLA-C | FLJ45422 | Hs.656020 | A_24_P110012 | NM_002117 |
| 136 | 60 | SERPINB6 | | Hs.519523 | A_24_P838743 | NM_004568 |
| 137 | 61 | POLE4 | | Hs.469060 | A_23_P154234 | NM_019896 |
| 138 | 62 | AKR7A2 | | Hs.571886 | A_23_P115356 | NM_003689 |
| 139 | 63 | EDARADD | | Hs.352224 | A_23_P503233 | NM_145861, NM_080738 |
| 140 | 64 | CARD9 | | Hs.694071 | A_23_P500433 | NM_052813 |
| 141 | 65 | RPL23 | | Hs.406300 | A_32_P30710 | NM_000978 |
| 142 | 142 | PBPP | | Hs.2164 | A_24_P675947 | NM_002704 |

1. A method for typing a cell isolate of an individual suffering from a pychiatric disorder, or at risk for suffering therefrom, the method comprising:
providing an RNA sample from a cell isolate from said individual;
determining RNA levels for a set of genes in said RNA sample, wherein said set of genes comprises at least two of the genes listed in Table 9; and
typing said isolate on the basis of the levels of RNA determined for said set of genes.

2. A method according to claim 1, comprising providing an RNA sample from said cell isolate after contacting said cell isolate with a stimulus.

3. A method according to claim 1 or 2, wherein said typing is based on a comparison with a reference.

4. A method according to claim 3, wherein said reference comprises RNA levels determined for said set of genes in a cell isolate prior to and/or after contacting said cell isolate with a stimulus.

5. A method according to claim 4, wherein said typing is based on the ratio of RNA levels determined in said RNA samples of a cell isolate prior to and after contacting said cell isolate with a stimulus.

6. A method according to any of the previous claims, wherein said pychiatric disorder comprises a depressive disorder.

7. A method according to any of claims 1-6, wherein said cell isolate comprises whole blood cells.

8. A method according to any of the previous claims, wherein said stimulus is selected from a cytokine, a lipopolysaccharide (LPS), a hormone, and a neurotransmitter.

9. A method according to claim 8, wherein said stimulus is LPS.

10. A method according to any of claims 1-9, wherein said set of genes comprises KRT23, CAPRIN, PLSCR1, PROK2, ZBTB16, TMEM4, CLEC4A, MLC1.

11. A method according to any of claims 1-10, wherein said set of genes comprises all of the genes listed in Table 9.

12. A method according to any of the previous claims, wherein said typing allows prognosticating syndrome severity, and/or a response to medical treatment.

13. A method according to claim 12, wherein said response to medical treatment comprises response to a selective serotonin reuptake inhibitor.

14. A set of probes for typing a cell isolate of an individual suffering from a depressive disorder or suspected of suffering therefrom, wherein said set of probes comprises probes specific for at least two of the genes listed in Table 9.

15. (canceled)

16. A set of primers for typing a cell isolate of an individual suffering from a depressive disorder or suspected of suffering therefrom, wherein said set of primers comprises primers specific for at least two of the genes listed in Table 9.

17. (canceled)

18. A method according to claim 1, wherein determining RNA levels for a set of genes in said RNA sample utilizes a set of probes comprising probes specific for at least two of the genes listed in Table 9.

19. A method according to claim 1, wherein determining RNA levels for a set of genes in said RNA sample utilizes a set of primers comprising primers specific for at least two of the genes listed in Table 9.

20. A method according to any one of claim 1, 18 or 19, wherein typing said isolate aids in prognosticating syndrome severity and/or a response to medical treatment for an individual suffering from a depressive disorder.
